SEQUENCE LISTING (17 sequences)

SEQ ID NO: 1 - DNA, 18 bp, Artificial Sequence
  Synthetically generated oligonucleotide
ggaagaaagtcgtggatt

SEQ ID NO: 2 - DNA, 19 bp, Artificial Sequence
  Synthetically generated oligonucleotide
ctggacttccagaagaaca

SEQ ID NO: 3 - protein, 235 aa, Homo sapiens
MGIGKSKINSCPLSLSWGKRHSVDTSPGYHESDSKKSEDLSLCNVAEHSNTTEGPTGKQEGAQSVEEMFEEEAEEEVFLKFVILHAEDDTDEALRVQNLLQDDFGIKPGIIFAEMPCGRQHLQNLDDAVNGSAWTILLLTENFLRDTWCNFQFYTSLMNSVNRQHKYNSVIPMRPLNNPLPRERTPFALQTINALEEESRGFPTQVERIFQESVYKTQQTIWKETRNMVQRQFIA

SEQ ID NO: 4 - protein, 17 aa, Homo sapiens
GIGKSKINSCPLSLSWG

SEQ ID NO: 5 - protein, 17 aa, Mus musculus
GVGKSKLDKCPLSWHKK

SEQ ID NO: 6 - protein, 232 aa, Mus musculus
MGVGKSKLDKCPLSWHKKDSVDADQDGHESDSKNSEEACLRGFVEQSSGSEPPTGEQDQPEAKGAGPEEQDEEEFLKFVILHAEDDTDEALRVQDLLQNDFGIRPGIVFAEMPCGRLHLQNLDDAVNGSAWTILLLTENFLRDTWCNFQFYTSLMNSVSRQHKYNSVIPMRPLNSPLPRERTPLALQTINALEEESQGFSTQVERIFRESVFERQQSIWKETRSVSQKQFIA

SEQ ID NO: 7 - protein, 142 aa, Homo sapiens
SRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVN

SEQ ID NO: 8 - protein, 147 aa, Homo sapiens
NIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWAN

SEQ ID NO: 9 - protein, 149 aa, Homo sapiens
AGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRR

SEQ ID NO: 10 - protein, 149 aa, Homo sapiens
FKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHK

SEQ ID NO: 11 - protein, 140 aa, Homo sapiens
TLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTR

SEQ ID NO: 12 - protein, 139 aa, Homo sapiens
PTHASDSGSSRWSKDYDVCVCHSEEDLVAAQDLVSYLEGSTASLRCFLQLRDATPGGAIVSELCQALSSSHCRVLLITPGFLQDPWCKYQMLQALTEAPGAEGCTIPLLSGLSRAAYPPELRFMYYVDGRGPDGGFRQV

SEQ ID NO: 13 - protein, 162 aa, Homo sapiens
FPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKE

SEQ ID NO: 14 - protein, 147 aa, Homo sapiens
SVEEMFEEEAEEEVFLKFVILHAEDDTDEALRVQNLLQDDFGIKPGIIFAEMPCGRQHLQNLDDAVNGSAWTILLLTENFLRDTWCNFQFYTSLMNSVNRQHKYNSVIPMRPLNNPLPRERTPFALQTINALEEESRGFPTQVERIF

SEQ ID NO: 15 - protein, 146 aa, mus musculus
AKGAGPEEQDEEEFLKFVILHAEDDTDEALRVQDLLQNDFGIRPGIVFAEMPCGRLHLQNLDDAVNGSAWTILLLTENFLRDTWCNFQFYTSLMNSVSRQHKYNSVIPMRPLNSPLPRERTPLALQTINALEEESQGFSTQVERIF

SEQ ID NO: 16 - DNA, 708 bp, Homo sapiens
atgggtatcgggaagtctaaaataaattcctgccctctttctctctcttggggtaaaaggcacagtgtggatacaagtccaggatatcatgagtcagattccaagaagtctgaagatctatccttgtgtaatgttgctgagcacagcaatacaacagaggggccaacaggaaagcaggagggagctcagagcgtggaagagatgtttgaagaagaagctgaagaagaggtgttcctcaaatttgtgatattgcatgcagaagatgacacagatgaagccctcagagtccagaatctgctacaagatgactttggtatcaaacccggaataatctttgctgagatgccatgtggcagacagcatttacagaatttagatgatgctgtaaatgggtctgcatggacaatcttattactgactgaaaactttttaagagatacttggtgtaatttccagttctatacgtccctaatgaactccgttaacaggcagcataaatacaactctgttatacccatgcggcccctgaacaatccccttccccgagaaaggactccctttgccctccaaaccatcaatgccttagaggaagaaagtcgtggatttcctacacaagtagaaagaatttttcaggagtctgtgtataagacacaacaaactatatggaaagagacaagaaatatggtacaaagacaatttattgcctga

SEQ ID NO: 17 - DNA, 699 bp, Mus musculus
atgggtgttgggaagtctaaactagataagtgccccctttcttggcataaaaaagacagtgtggatgccgatcaagacggccatgagtcagactccaagaattctgaggaagcctgcttgcgtggttttgtggagcagagcagtggatcagagccaccaacaggagagcaggaccaacctgaggcaaagggggcggggcctgaggagcaagatgaagaagagttcctcaaatttgtgatacttcatgcagaagatgacaccgacgaggccctcagagtccaggatctactgcaaaacgactttggtatcaggccggggatcgttttcgccgagatgccgtgcggaagactgcatttgcagaatctagacgatgcggtcaatgggtcggcctggaccatcttgttactgactgagaacttcctaagagacacctggtgtaacttccagttctacacttccctgatgaattctgtgagcaggcagcacaagtacaactccgtcatacccatgcggcccctgaacagccctcttccccgggaacggactcccttggcgctgcaaaccatcaatgccttagaggaagaaagtcaaggcttttccacacaagtggagagaattttccgggagtctgtatttgagaggcaacaaagtatatggaaagagacacgaagtgtgtcgcagaaacagttcattgcctga

(19) United States
(12) Patent Application Publication
(10) Pub. No.: US 2005/0158799 A1
(43) Pub. Date: Jul. 21, 2005

(54) TRIF-related adaptor molecule (TRAM) and uses thereof

(76) Inventors: Katherine A. Fitzgerald, Cambridge, MA (US); Daniel C. Rowe, Walpole, MA (US); Douglas T. Golenbock, Wellesley, MA (US)

Correspondence Address: FISH & RICHARDSON PC, 225 FRANKLIN ST, BOSTON, MA, 02110, US

(21) Appl. No.: 10/968,598

(22) Filed: Oct. 18, 2004

Related U.S. Application Data
(60) Provisional application No. 60/512,364, filed on Oct. 17, 2003.

(51) Int. Cl. G01N 33/53; G01N 33/567; C07H 21/04; C07K 14/715; C07K 16/28; C12N 5/06

(52) U.S. Cl. 435/7.2; 435/69.1; 435/334; 435/320.1; 530/350; 530/388.22; 536/23.5

(57) ABSTRACT

A Toll-IL-1-resistance (TIR) domain-containing adaptor-inducing IFN-β (TRIF)-related adaptor molecule (TRAM) has been identified. TRAM acts specifically in the TLR4 signaling pathway. The invention includes compounds useful for modulating TLR signaling by modulating the effects of TRAM.

### CLAIM OF PRIORITY

This application claims priority under 35 USC §119(e) to U.S. Provisional Patent Application Ser. No. 60/512,364, filed on Oct. 17, 2003, the entire contents of which are hereby incorporated by reference.

### FEDERALLY SPONSORED RESEARCH OR DEVELOPMENT

This invention was made pursuant to Grant Nos. DK50305, GM54060, GM63244, and IR21 A1055453701. The U.S. Government has certain rights in this invention.

### TECHNICAL FIELD

This invention relates to modulation of immunity, using Toll-IL-1-resistance domain-containing adapter-inducing IFN-b-related adapter molecule (TRAM).

### BACKGROUND

The Toll-Like Receptor (TLR) family is the essential recognition and signaling component of mammalian host defense (Medzhitov et al., Nature 388: 394-97, 1997; Akira, Adv. Immunol. 78: 1-56, 2001; Dunne and O'Neill, Sci STKE 171: re3, 2003). At least ten TLRs have been cloned in mammals, which recognize molecular products derived from all the major classes of pathogens (Medzhitov, et al., 1997, supra; Akira, 2001, supra; Dunne and O'Neill, 2003, supra. Toll signaling to nuclear factor-kappaB (NF-κB) originates from the conserved Toll-IL-1-Resistance (TIR) domain, which mediates recruitment of the TIR domain-containing adapter molecule, myeloid differentiation factor 88 (MyD88) (Muzio et al., Science 278: 1612-15, 1997), a critical adapter molecule utilized by most TLRs (Janssens and Beyaert, Trends Biochem. Sci. 27: 474-82, 2002). The recruitment of MyD88 to proximal TIR domains of activated TLRs allows for the interaction and activation of the IRAK-family members (Cao et al., Science 271: 1128-31, 1996; Li, et al., Proc. Natl. Acad. Sci. USA 99: 5567-5572, 2002), and the subsequent activation of tumor necrosis factor receptor-associated factor-6 (TRAF-6) (Cao, et al., Nature 383: 443-46, 1996). These events, at a minimum, result in NF-κB activation via the I-kappa B Kinase (IKK)α-β-γ complex (Karin and Ben-Neriah, Annu. Rev. Immunol. 18: 621-63, 2000).

Most of the TLRs appear to be dependent on the expression of MyD88 for all of their functions, TLR3 and TLR4 are unique in their ability to activate MyD88-independent responses (Kawai, et al., Immunity 11: 115-22, 1999; Kaisho et al., J. Immunol. 166: 5688-94, 2001; Toshchakov et al., Nat. Immunol. 3: 392-98, 2002; Oshiumi et al., Nat. Immunol. 4(2): 161-7, 2003). A feature of MyD88-independent signaling is the induction of a dendritic cell maturation pathway, and the induction of the type 1 interferon (IFN-β) (Kaisho and Akira, Trends Immunol. 22: 78-83, 2001; Kawai et al, J. Immunol. 167: 5887-94, 2001; Toshchakov, 2002, supra Yamamoto et al., J. Immunol. 169: 6668-72, 2002; Oshiumi, 2003, supra. The transcription enhancer of the IFN-β promoter binds NF-κB, interferon regulatory factor 3 (IRF-3) and activating transcription factor-2 (ATF-2)/c-Jun. While all TLRs activate NF-κB and ATF2-c-Jun, not all TLRs induce IFN-β, because not all TLRs induce IRF-3 activation.

### SUMMARY

The invention is based, at least in part, on the identification of “Toll-IL-1-resistance domain-containing adapter-inducing IFN-β-related adapter molecule” (TRAM), a polypeptide that is involved in the TLR4 signaling pathway.

The invention includes isolated polypeptides including the amino acid sequence of murine TRAM or human TRAM, as described herein. In some embodiments, the isolated polypeptide includes the amino acid sequence of SEQ ID NO:3 or 6, or an active fragment thereof. In some embodiments, the active fragment has one or more activities of the full length TRAM, e.g., it can bind to one or more of Toll-IL-1-resistance domain-containing adaptor-inducing IFN-J (TRIF), Toll-Like Receptor 4 (TLR4), CREB-Binding Polypeptide (CBP), or MyD88 adaptor-like (Mal); can form a complex with Mal and Myeloid Differentiation Primary Response Gene 88 (MyD88); and/or can induce nuclear factor kappa B (NFkB) or interferon regulatory factor 3 (IRF-3) dependent gene expression in a cell, in response to stimulation of a TLR4 receptor expressed in the cell. In some embodiments, the active fragment can inhibit one or more activity of the full length TRAMpolypeptide.

In some embodiments, the invention includes isolated nucleic acids encoding TRAM polypeptides and fragments thereof, e.g., the nucleic acid sequence of murine TRAM or human TRAM, as described herein. In some embodiments, the nucleic acids have the sequence of SEQ ID NO:16 or 18, or a fragment therof. The invention also includes oligonucleotides including at least about 15 consecutive nucleotides of SEQ ID NO:16 or 18.

In part, the invention relates to methods of identifying candidate compounds that modulate an interaction between TRAM and a TRAM-effector. The methods include providing a sample including a TRAM polypeptide and a TRAM-effector, contacting the sample with a test compound, and determining the level of interaction between the TRAM and TRAM-effector in the presence of the test compound compared to the level of interaction in a control sample, such that a difference in the level of interaction indicates that the test compound is a candidate compound for modulating the interaction between TRAM and a TRAM-effector. The test compound can increase or decrease the amount of the interaction. The test compound can be an antibody, e.g., one that specifically binds to a site that includes at least one of Cysteine 117 (C117) or Proline 116 (P116) of human TRAM (SEQ ID NO:3). A TRAM-effector can be Toll/IL-1 receptor-domain-containing adaptor inducing IFN-beta (TRIF), MyD88 Adaptor-Like (Mal), Toll-Like Receptor 4 (TLR4), CREB-Binding Protein (CBP), Myeloid Differentiation Primary Response Gene 88 (MyD88), or p300. In some cases, the TRAM and the TRAM-effector are in a cell, e.g., the test sample includes one or more cells that possess TRAM and a TRAM-effector (e.g., the cells express endogenous or exogenous TRAM and/or a TRAM-effector, or have TRAM and/or a TRAM-effector added to them). In some embodiments, the interaction is binding, e.g., binding of TRAM to TLR4, TRAM to Mal, Mal to MyD88, or TRIF, for example.

Other methods for identifying candidate compounds that can modulate TRAM signaling include providing cells that express TRAM, contacting the cells with a test compound, and determining TRAM polypeptide localization in the cells. A difference in the TRAM polypeptide localization in the presence of the test compound, as compared to a control, indicates that the test compound is a candidate compound for modulating TRAM signaling. The TRAM can be a fluorescent TRAM fusion polypeptide. In some cases the test compound is an inhibitor of myristoylation. In some embodiments, the difference in localization is an increase in cytoplasmic localization of the TRAM, and/or a decrease in membrane localization. In some cases, the test sample includes or is a cell, e.g., the test sample includes one or more cells that posses TRAM (e.g., cells that express endogenous or exogenous TRAM, or have TRAM added to them).

Alternatively, methods for identifying candidate compounds that can modulate TRAM signaling can include providing a test sample including a TRAM polypeptide that contains the TRAM myristoylation site (e.g., SEQ ID NO:4) and a compound that can myristoylate TRAM, e.g., myristoylCoA:polypeptide N-myristoyltransferase (NMT) contacting the test sample with a test compound, and determining the level of myristoylation of TRAM in the test sample. A decrease in myristoylation of TRAM in the test sample, e.g., as compared to a control, indicates that the test compound is a candidate compound for modulating TRAM signaling. In some cases, the test sample is a cell, e.g., the test sample includes one or more cells that poss TRAM and a compound that can myristoylate TRAM (e.g., cells that express endogenous or exogenous TRAM and/or a compound that can myristoylate TRAM, or have TRAM and/or a compound that can myristoylate TRAM added to them).

The invention also features methods for determining whether a test compound can modulate TRAM signaling. The methods can include providing a test sample including cells that can exhibit TRAM signaling, contacting the cells with an inducer of TRAM signaling, e.g., lipopolysaccharide (LPS) or gram-negative bacteria, or other TLR4 agonist, and a test compound, and determining the amount of expression or activity of an indicator of TRAM signaling, e.g., IRF-3 or a type I IFN such as IFNα or IFN β, in the test sample. A difference in the amount of expression or activity of the indicator of TRAM signaling in the test sample, as compared to the amount of indicator of TRAM signaling expression or activity in a control cell that was not contacted with the test compound, indicates that the test compound can modulate TRAM signaling.

The methods described herein for identifying compounds that modulate TRAM signalling can also be considered methods for identifying compounds that modulate TLR4 signalling, as modulators of TRAM signalling will also likely modulate TLR4 signalling. Compounds that decrease TLR4/TRAM signalling can be used to treat inflammatory conditions in a subject, e.g., by administering a therapeutically effective amount of such a compound.

In another embodiment, the invention relates to methods of modulating the ability of a cell to effect TLR4 signaling, e.g., to signal in response to a TLR4 agonist such as LPS. The methods include providing a cell that can undergo TLR4 signaling and contacting the cell with an amount of a compound that modulates TRAM expression or activity in an amount sufficient to modulate expression or activity of TRAM, thereby modulating the ability of the cell to effect TLR4 signaling. In some cases the compound is an siRNA or an antibody. The compound may modulate myristoylation of TRAM. The compound, in some cases, increases TLR4 signaling. In other cases, it decreases TLR4 signaling. TLR4 signaling can be detected by assaying IFN-β activation, RANTES (regulated on activation, normal T cell expressed and secreted) secretion, or induction of γ interferon-inducible polypeptide 10 (IP10), IRF1, or IFIT1 (interferon-induced polypeptide with tetratricopeptide repeats 1).

The invention also relates to methods of detecting TLR signaling. The methods include providing a cell, e.g., a bone marrow-derived macrophage, that expresses a TLR, contacting the with an inducer of TLR signaling, and detecting a level of secretion of RANTES, activation of IFN-β, or the level of expression of IP10. In some embodiments, the TLR is TLR3 or TLR4. The level of secretion of RANTES, activation of IFN-J, or expression of IP10 indicates the presence of TLR signalling in the cell. In some embodiments, the method includes comparing the level of secretion of RANTES, activation of IFN-J, or expression of IP10 in the absence of the inducer of TLR signaling. In some embodiments, the methods include contacting the cell with a test compound and determining the effect of the test compound on TLR signaling in the cell.

In another embodiment, the invention relates to methods of ameliorating an inflammatory response in a cell. The methods include providing a cell that is susceptible to or undergoing an inflammatory response, and contacting the cell with a compound that decreases TRAM expression or activity in an amount sufficient to decrease an inflammatory response, e.g., a compound identified by a method described herein. Also included are methods of decreasing or preventing an inflammatory response in a subject. These methods include administering to the subject a therapeutically effective amount of at least one compound that decreases TRAM expression or activity in an amount sufficient to decrease the inflammatory response in the subject. In some embodiments, the compound is a TRAM antisense oligonucleotide, TRAM siRNA, TRAM morpholino oligonucleotide, anti-TRAM antibody, or a TRAM dominant negative polypeptide. In some embodiments, the methods include identifying a subject having or susceptible to an inflammatory response.

In some embodiments, the invention relates to antibodies that specifically bind to a TRAM polypeptide, e.g., antibodies that specifically binds to a TRAM polypeptide that includes at least one of TRAM-C117, TRAM-P116, or the myristoylation site of TRAM.

A molecule that “specifically” binds to a particular entity, e.g., a TRAM polypeptide, binds to that entity in a sample, e.g., a biological sample, but does not substantially recognize or bind to other molecules in the sample.

A “polypeptide” in a chain of amino acids regardless of length or post-translational modifications. As used herein, the term “TRAM” means a TRAM polypeptide unless otherwise indicated.

A “TRAM-effector” is a molecule (e.g., a polypeptide) that co-immunoprecipitates with TRAM and that is expressed in a cell, or that functions in the same pathway as TRAM. Examples of TRAM-effectors are Mal (MyD88 adaptor-like; also known as TIR Domain-Containing Adaptor Polypeptide (TIRAP); OMIM 606252), TLR4 (TOLL-Like Receptor 4; OMIM 603030), CBP (CREB-Binding Polypeptide; CREBBP; OMIM 600140), MyD88 (Myeloid Differentiation Primary Response Gene 88; OMIM 602170), or p300 (also known as E1A-Binding Polypeptide, 300-KD; EP300, OMIM 602700), RANTES (regulated on activation, normal T cell expressed and secreted; also known as chemokine CC motif ligand 5, (CCL5), small inducible cytokine A5 (SCYA5), T cell-specific RANTES, T cell-specific polypeptide p228, or TCP228; OMIM 187011), and IP-10 (γ interferon-inducible polypeptide 10, also known as chemokine, cxc motif, ligand 10 (CXCL10), small inducible cytokine subfamily B, member 10, (SCYB10), INP10, interferon-gamma-induced factor; OMIM 147310) and TRIF (TIR Domain-Containing Adaptor Inducing Interferon-Beta; also known as TIR Domain-Containing Adaptor Molecule 1, TICAM1; OMIM 607601).

“Subject,” as used herein, refers to a mammal, e.g., a human, or to an experimental animal (e.g., disease) model. The subject can be a non-human animal, e.g., a mouse, rat, cat, dog, guinea pig, horse, cow, pig, goat, or other domestic animal. An experimental animal as described herein can be a TRAM knockout animal, e.g., as described in Yamamoto et al., Nature Immun. 4: 1144-50, 2003. The subject can be, e.g., a human subject in a clinical trial.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DESCRIPTION OF DRAWINGS

FIGS. 1A and 1B are graphs depicting the results of ELISA analysis of the amount of RANTES (Regulated on Activation, Normal T cell Expressed and Secreted) secretion from bone marrow derived macrophages from wild type and MyD88-deficient mice.

FIG. 1C is triptych of reproductions of autoradiograms of nuclear extracts from wild type and MYD88-deficient macrophages stimulated with LPS, Malp-2 (a mycoplasmal lipopeptide), or dsRNA and subjected to electrophoretic mobility shift assay using a labeled ISRE (interferon-stimulated-response-element) consensus sequence (ISG-15) as a probe. Activated ISRE DNA-binding complexes were preincubated with polyclonal antibody to IRF-3 or two control antibodies before incubation with the ISRE probe (right panel).

FIGS. 1D and 1E are bar graphs depicting the results of experiments in which the relative level of stimulation of a GAL-4 fusion polypeptide by increasing concentrations of dsRNA (poly IC) or by LPS.

FIG. 2A is a representation of an alignment of TIR domains of TRAM, TRIF, MyD88 and Mal with TLR1, TLR2, TLR3, and TLR4. The amino acid shades are based on their physico-chemical properties; yellow=small, green=hydrophobic, turquoise=aromatic, blue=positively charged and red=negatively charged.

FIG. 2B is a pair of bar graphs depicting the relative stimulation of HEK293 cells transfected as in FIGS. 1D-E (above), and cotransfected with TRAM or TRIF.

FIG. 3A is a set of photomicrographs of IRF-3-GFP-expressing HEK293 cells transiently transfected with TRAM, TRIF, or pCDNA3.1 and visualized using confocal microscopy.

FIG. 3B is a reproduction of a set of Western blots in which 293T cells were transfected with Flag-TRAM with or without a plasmid encoding IRF-3 (untagged) as indicated (above the blots). Whole cell lysates were immunoprecipitated with anti-IRF-3, anti-Flag, or anti-CBP and the immunoprecipitated complexes immunoblotted for Flag-tagged TRAM and IRF-3. Whole cell lysates (WCL) were also analyzed for Flag-tagged polypeptides.

FIG. 3C is a bar graph depicting the relative stimulation of RANTES in HEK293 cells that were transfected with the RANTES luciferase reporter gene and TRAM and cotransfected with increasing concentrations of IKKε-k38a, TBK1-k38a, or IRF-3-ΔN at 10, 20, 30, 40, 60, or 80 ng.

FIG. 4A is a bar graph depicting the results of experiments in which HEK293 cells were transfected with a RANTES reporter construct and cotransfected with TRAM, TRAM-TIR, or TRAM mutants in which the cysteine at position 117 is changed to a histidine (TRAM-C117H), or the proline at position 116 is changed to a histidine (TRAM-P116H). Relative stimulation of RANTES was assayed.

FIGS. 4B-4G are bar graphs depicting the results of experiments in which HEK293 cell lines expressing TLR4/MD2 (4C, 4E, and 4G) or TLR3 (4B, 4D, and 4F) were transfected with a luciferase reporter gene containing the Gal4 upstream activation sequence and cotransfected with Gal4-DBD, Gal4-IRF-3 (4D-E), or Gal4-IRF-7 (4D-E), or the RANTES luciferase reporter gene (4F-G) as well as TRAM-C117H or TRIF-ΔNΔC. After incubation, the cells were stimulated with LPS (4C and 4G), dsRNA (poly IC, 4B and 4F), or not treated (medium), then incubated, and luciferase activity measured.

FIG. 5A is a bar graph depicting the relative stimulation of NF-κB in HEK293 cells transfected with an NF-κB reporter construct and co-transfected with TRAM, TRAM-TIR, TRAM-P116H, or TRAM-C117H.

FIGS. 5B-G are bar graphs depicting the results of experiments in which various TLR-expressing HEK293 stable cell lines were transfected with an NF-κB reporter gene and co-transfected with increasing concentrations of TRAM-C117H. One day after transfection, TLR-expressing cells were stimulated with Malp-2 (2 nM) (5B), dsRNA (100 μg/ml poly I:C, 5C), LPS (10 ng/ml, 5E), R-848 (10 μM, 5D), IL1 (10 ng/ml, 5F), TNFα (10 ng/ml, 5G) or left untreated (medium) for eight hours,
and luciferase reporter gene activity was measured.

FIG. 6A is a bar graph depicting relative stimulation of RANTES in HEK293 cells transfected with RANTES luciferase reporter gene, TRAM, or TRIF expressing constructs and co-transfected with TRIF-ΔNΔC or TRAM-C117H.

FIG. 6B is a set of immunoblots depicting the results of experiments in which 293T cells were transfected with TRAM-CFP or TRIF-CFP and co-transfected with Flag-Mal, Flag-Mal-P125H or Flag-TRIF. Whole cell lysates were harvested 48 hours later, and immunoprecipitated with anti-GFP antibody (which also immunoprecipitates cyan fluorescent polypeptide; CFP or yellow fluorescent polypeptide; YFP). Immunoprecipitated complexes were resolved by SDS-PAGE and immunoblotted for Flag-tagged adapters. Whole cell lysates (WCL) were also analyzed for CFP- and Flag-tagged polypeptides by immunoblotting.

FIG. 6C is a set of immunoblots depicting the results of experiments in which stable TLR4^{YFP} or TLR3^{YFP}-expressing HELA cells were transfected with a plasmid encoding Flag-Mal, Flag-TRAM, or Flag TRAM-C/H. 48 hours later, whole cell lysates were immunoprecipitated with anti-GFP antibody and immunoprecipitated complexes immunoblotted for Flag-tagged adapters. Western blotting of lysates demonstrates expression of stable TLRs and transfected adapter polypeptides.

FIG. 7A is a set of three graphs depicting the results of experiments in which 293T cells were plated and transfected with plasmids encoding TRAM-CFP, TRIF-CFP, or Mal-CFP and co-transfected with siRNA-TRAM or Lamin A/C as indicated. After incubation, CFP fluorescence was measured by flow cytometry.

FIGS. 7B-7C are bar graphs depicting experiments in which U373/CD14 or TLR3-expressing HEK293 cells were transfected with a RANTES reporter gene and co-transfected with siRNA duplexes as indicated for 36 hours. Cells were then stimulated for 8 hours with LPS or dsRNA and luciferase reporter gene activity was measured.

FIG. 8 is a drawing of a model of TRAM activity.

FIG. 9 is a set of bar graphs illustrating RANTES induction in peritoneal macrophages isolated from wild type (C57/BL6, black bars) or TRAM knockout (TRAM −/−, gray bars) mice, stimulated with LPS, heat-killed E. Coli (gram negative bacteria), heat-killed group B streptococcus (gram positive), R848 (a TLR7 agonist, GL Synthesis, Worcester, Mass.), Sendai Virus (a non-TLR activating pathogen, Charles River Laboratories, Wilmington, Mass.), or CpG DNA (a TLR9 agonist, MWG Synthesis, High Point, N.C.).

FIGS. 10A-10C are sets of three fluorescent photomicrographs illustrating the subcellular localization of Mal-CFP (10A), MyD88-CFP (10B) and TRAM-CFP (10C) fusion constructs in cells cotransfected with a TLR4-YFP fusion construct. The left panels show the CFP signal, representing the fusion constructs alone; the middle panels show the YFP signal, representing TLR4; and the right panels show the overlay of the two signals.

FIGS. 11A-11D are sets of three fluorescent photomicrographs illustrating the subcellular localization of TRAM-CFP (11A), the TRAM myristoylation mutant TRAM G2A-CFP (11B), MyD88-CFP (11C), and the MyD88 mutant with the myristoylation sequence from TRAM, Myr-MyD88-CFP (11D) fusion constructs, in cells co-transfected with a fusion polypeptide of Src kinase fused to YFP (Myr-YFP). The left panels show the CFP signal, representing the fusion constructs alone; the middle panels show the YFP signal, representing the Myr-YFP, and the right panels show the overlay of the two signals.

FIG. 12 is a pair of autoradiograms. The top panel shows the incorporation of tritiated [³H] myristic acid into TRAM and the Myr-MyD88 mutant, and the bottom panel shows a Western blot of whole cell lysates probed with anti-GFP, showing that the fluorescent fusion proteins were expressed.

FIGS. 13 and 14 are bar graphs illustrating the effect of transfection with increasing concentrations of expression vectors for wild type TRAM, TRAM G2A non-myristoylated mutant, or TRAM C117H dominant negative on the induction of IRF-3 (13) or NFkB (14) dependent gene expression.

FIG. 15 represents the amino acid and nucleic acid sequences of mouse and human TRAM. In the human sequence, P116 and C117 are bold and underlined.

### DETAILED DESCRIPTION

The TRIF-related adapter molecule (TRAM) is described herein. It was found that TRAM contains a TIR domain and participates in TLR4 signaling. Like TRIF, TRAM activates IRF-3-, IRF-7-, and NF-κB-dependent signaling pathways. TLRs-3 and -4 activate these pathways to induce IFN-α/β, RANTES, and IP-10 expression independently of the adapter polypeptide MyD88. Knockout, dominant-negative and siRNA studies described herein demonstrate that TRIF functions downstream of both the TLR3 (which signals in response to double-stranded RNA) and TLR4 (which signals in response to lipopolysaccharide) signaling pathways, while the function of TRAM is restricted to the TLR4 pathway. TRAM interacts with TRIF, Mal (MyD88-adapter-like polypeptide), CBP, p300, and TLR4, but not with TLR3. The results described herein suggest that TRIF and TRAM both function in LPS/TLR4 signaling to

TRAM Nucleic Acids and Polypeptides

The term “isolated or purified nucleic acid molecule” includes nucleic acid molecules that are separated from other nucleic acid molecules that are present in the natural source of the nucleic acid. For example, with respect to genomic DNA, the term “isolated” includes nucleic acid molecules that are separated from the chromosome with which the genomic DNA is naturally associated. Generally, an “isolated” nucleic acid is free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of 5′ and/or 3′ nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent hybridization conditions are hybridization in 0.5M sodium phosphate, 7% SDS at 65° C., followed by one or more washes at 0.2×SSC, 1% SDS at 65° C. Other stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. Included herein is an isolated nucleic acid molecule that hybridizes under stringent conditions to the sequence of Genbank accession no. AY268050 (mouse) or AY232653 (human), or the complement thereof, and corresponds to a naturally-occurring nucleic acid molecule, or the complement thereof.

As used herein, a “naturally-occurring” nucleic acid molecule refers to an RNA or DNA molecule having a nucleotide sequence that occurs in nature (e.g., encodes a natural polypeptide).

As used herein, the terms “gene” and “recombinant gene” refer to nucleic acid molecules that include an open reading frame encoding a TRAM polypeptide, preferably a mammalian TRAM polypeptide, and can further include non-coding regulatory sequences, and introns.

An “isolated” or “purified” polypeptide or polypeptide is substantially free of cellular material or other contaminating polypeptides from the cell or tissue source from which the polypeptide is derived, or substantially free from chemical precursors or other chemicals when chemically synthesized. In one embodiment, the language “substantially free” means preparation of TRAM polypeptide having less than about 30% (by dry weight), of non-TRAM polypeptide (also referred to herein as a “contaminating polypeptide”), or of chemical precursors or non-TRAM chemicals; in some embodiments, the preparation has less than about 20%, 10%, 5%, or less of non-TRAM polypeptide. When the TRAM polypeptide or biologically active portion thereof is recombinantly produced, it is also generally substantially free of culture medium, i.e., culture medium represents less than about 20%, e.g., less than about 10% or 5% of the volume of the polypeptide preparation. The invention includes isolated or purified preparations of at least 0.01, 0.1, 1.0, and 10 milligrams in dry weight.

A “non-essential” amino acid residue is a residue that can be altered from the wild-type sequence of TRAM (e.g., Genbank accession no. AY268050 (mouse) or AY232653 (human)) without abolishing and generally, without substantially altering a biological activity, whereas an “essential” amino acid residue results in such a change. For example, certain amino acid residues present in the functional domains, e.g., the TIR or myristoylation domains, are predicted to be particularly un-amenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a TRAM polypeptide is generally replaced with another amino acid residue from the same side chain family. Alternatively, in another embodiment, mutations can be introduced randomly along all or part of a TRAM coding sequence, such as by saturation mutagenesis, and the resultant mutants can be screened for TRAM biological activity to identify mutants that retain activity. Following mutagenesis of Genbank accession no. AY268050 (mouse) or AY232653 (human), the encoded polypeptide can be expressed recombinantly and the activity of the polypeptide can be determined.

As used herein, a “biologically active portion” of a TRAM polypeptide includes a fragment of a TRAM polypeptide that participates in an interaction between a TRAM molecule and a non-TRAM molecule (e.g., a TRAM-effector). Biologically active portions of a TRAM polypeptide include peptides including amino acid sequences sufficiently homologous to or derived from the amino acid sequence of the TRAM polypeptide, which include fewer amino acids than the full-length TRAM polypeptides, and exhibit at least one activity of a TRAM polypeptide, e.g., co-immunoprecipitation with Mal, TRIF or TLR4, activation of the NFkB and/or IRF-3 pathways as described herein, or any of the other activities described herein. Typically, biologically active portions include a domain or motif with at least one activity of the TRAM polypeptide, e.g., the myristoylation site that confers the ability to localize to a membrane. A biologically active portion of a TRAM polypeptide can be a polypeptide that is, for example, 10, 25, 50, 100, 150, 200 or more amino acids in length. Biologically active portions of a TRAM polypeptide can be used as targets for developing agents that modulate a TRAM mediated activity, e.g., activation of the innate immune system, e.g., by disrupting the interaction of TRAM with TLR4, TRIF, or Mal, or formation of complexes including TRAM and TRAM-effector polypeptides, e.g., a complex including TRAM, Mal, and MyD88.

Calculations of homology or sequence identity between sequences (the terms are used interchangeably herein) are performed as follows.

To determine the percent identity of two amino acid or nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 60%, e.g., at least 70%, 80%, 90%, or 100% of the length of the reference sequence (e.g., when aligning a second sequence to the TRAM amino acid sequence of Genbank accession no. AY268050 (mouse) or AY232653 (human) and having at least 60% 70%, 80%, or 90% amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using the Needleman and Wunsch (J. Mol. Biol. 48: 444-53, 1970) algorithm that has been incorporated into the GAP program in the GCG software package (available on the internet at gcg.com), using a Blossum 62 scoring matrix with a gap penalty of 12, a gap extend penalty of 4, and a frameshift gap penalty of 5.

The nucleic acid and polypeptide sequences described herein can be used as a “query sequence” to perform a search against public databases to, for example, identify other family members or related sequences. Such searches can be performed using the NBLAST and/or XBLAST programs (version 2.0) of Altschul et al., J. Mol. Biol. 215: 403-10, 1990. BLAST nucleotide searches can be performed with the NBLAST program, score=100, wordlength=12 to obtain nucleotide sequences homologous to the TRAM nucleic acid molecules described herein. BLAST polypeptide searches can be performed with the XBLAST program, score=50, wordlength=3 to obtain amino acid sequences homologous to TRAM polypeptide molecules described herein. To obtain gapped alignments for comparison purposes, Gapped BLAST can be utilized as described in Altschul et al., Nucleic Acids Res. 25: 3389-3402, 1997. When utilizing BLAST and Gapped BLAST programs, the default parameters of the respective programs (e.g., XBLAST and NBLAST) can be used. See, e.g., the National Center for Biotechnology Information's website at ncbi.nlm.nih.gov.

The TRAM polypeptides described herein have an amino acid sequence sufficiently identical to the amino acid sequence of Genbank accession no. AY268050 (mouse) or AY232653 (human). The term “sufficiently identical” or “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 60% identity are defined herein as sufficiently or substantially identical, e.g., about 65%, 70%, 75%, 80%, 85%, 90%, 91%, 92%, 93%, 94%, 95%, 96%, 97%, 98% or 99% identity.

“Misexpression or aberrant expression,” as used herein, refers to a non-wild type pattern of gene expression, at the RNA or polypeptide level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

A “purified preparation of cells,” as used herein, refers to, in the case of plant or animal cells, an in vitro preparation of cells and not an entire intact plant or animal. In the case of cultured cells or microbial cells, it consists of a preparation of at least 50%, e.g., 70%, 80%, or 90%, of the subject cells, by weight.

Isolated TRAM Nucleic Acid Molecules

Isolated or purified nucleic acid molecules that encode TRAM polypeptides are described herein, e.g., a full length TRAM polypeptide or a fragment thereof, e.g., a biologically active portion of TRAM polypeptide. Also described are nucleic acid fragments suitable for use as hybridization probes, which can be used, e.g., to identify a nucleic acid molecule encoding a TRAM polypeptide as described herein, e.g., TRAM mRNA, and fragments suitable for use as primers, e.g., PCR primers for the amplification or mutation of TRAM nucleic acid molecules as described herein.

In one embodiment, an isolated nucleic acid molecule as described herein includes the nucleotide sequence shown in Genbank accession no. AY268050 (mouse) or AY232653 (human), or a portion of any of these nucleotide sequences. In one embodiment, the nucleic acid molecule includes sequences encoding the human TRAM polypeptide (i.e., “the coding region”), as well as 5′ untranslated sequences. Alternatively, the nucleic acid molecule can include only the coding region and, e.g., no flanking sequences that normally accompany the subject sequence. In another embodiment, an isolated nucleic acid molecule described herein includes a nucleic acid molecule that is a complement of the nucleotide sequence shown in Genbank accession no. AY268050 (mouse) or AY232653 (human), or a portion of any of these nucleotide sequences. In other embodiments, the nucleic acid molecule described herein is sufficiently complementary to the nucleotide sequence shown in Genbank accession no. AY268050 (mouse) or AY232653 (human) such that it can hybridize to the nucleotide sequence shown in Genbank accession no. AY268050 (mouse) or AY232653 (human), thereby forming a stable duplex.

In one embodiment, an isolated nucleic acid molecule as described herein includes a nucleotide sequence that is at least about 60%, e.g., 65%, 70%, 75%, 80%, 85%, 90%, 91%, 92%, 93%, 94%, 95%, 96%, 97%, 98%, 99%, or more identical to the entire length of the nucleotide sequence shown in Genbank accession no. AY268050 (mouse) or AY232653 (human), or a portion, generally of the same length, of any of these nucleotide sequences.

TRAM Nucleic Acid Fragments

A nucleic acid molecule described herein can include only a portion of the nucleic acid sequence of Genbank accession no. AY268050 (mouse) or AY232653 (human). For example, such a nucleic acid molecule can include a fragment that can be used as a probe or primer or a fragment encoding a portion of a TRAM polypeptide, e.g., an immunogenic or biologically active portion of a TRAM polypeptide. A fragment can comprise, e.g., those nucleotides of Genbank accession no. AY268050 (mouse) or AY232653 (human) that encode a TIR domain or a myristoylation site of TRAM. The nucleotide sequence determined from the cloning of the TRAM gene enables the generation of probes and primers designed for use in identifying and/or cloning other TRAM family members, or fragments thereof, as well as TRAM homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment that includes a nucleotide sequence encoding an amino acid fragment described herein. Nucleic acid fragments can encode a specific domain or site described herein or fragments thereof, particularly fragments thereof that are at least 100 amino acids in length. Fragments also include nucleic acid sequences corresponding to specific amino acid sequences described above or fragments thereof. Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a TRAM nucleic acid fragment can include a sequence corresponding to the TIR domain or a myristoylation site.

TRAM probes and primers are also provided. Typically a probe/primer is an isolated or purified oligonucleotide. The oligonucleotide typically includes a region of nucleotide sequence that hybridizes under stringent conditions to at least about 20, e.g., at least about 25, 30, 35, 40, 45, 50, 55, 60, 65, 75 or more consecutive nucleotides of a sense or antisense sequence of Genbank accession no. AY268050 (mouse) or AY232653 (human), or of a naturally occurring allelic variant or mutant of Genbank accession no. AY268050 (mouse) or AY232653 (human) (for example a mutation at position 116, 117, or G2 in the myristoylation site of TRAM).

In a preferred embodiment, the nucleic acid is a probe that is at least 5 or 10, and less than 200, more preferably less than 100, or less than 50, base pairs in length. It should be identical, or differ by 1, or less than 1, in 5 or 10 bases, from a sequence disclosed herein. If alignment is needed for this comparison the sequences should be aligned for maximum homology. “Looped” out sequences from deletions or insertions, or mismatches, are considered differences.

In another embodiment a set of primers is provided, e.g., primers suitable for use in a PCR, which can be used to amplify a selected region of a TRAM sequence, e.g., a domain, region, site, or other sequence described herein. The primers should be at least 5, 10, 20, 30, 35, 40 or 50 base pairs in length and less than 100, or less than 200, base pairs in length. The primers should be identical, or differs by one base from a sequence disclosed herein or from a naturally occurring variant.

A nucleic acid fragment can encode an antigenic region of a TRAM polypeptide described herein.

A nucleic acid fragment encoding a “biologically active portion of a TRAM polypeptide” can be prepared by isolating a portion of the nucleotide sequence of Genbank accession no. AY268050 (mouse) or AY232653 (human), which encodes a polypeptide having a TRAM biological activity (e.g., the biological activities of the TRAM polypeptides are described herein), expressing the encoded portion of the TRAM polypeptide (e.g., by recombinant expression in vitro), and assessing the activity of the encoded portion of the TRAM polypeptide. For example, a nucleic acid fragment encoding a biologically active portion of TRAM includes a myristoylation site. A nucleic acid fragment encoding a biologically active portion of a TRAM polypeptide, may comprise a nucleotide sequence that is greater than 300 or more nucleotides in length.

In preferred embodiments, a nucleic acid includes a nucleotide sequence that is about 300, 400, 500, 600, or more nucleotides in length, and hybridizes under stringent hybridization conditions to a nucleic acid molecule of Genbank accession no. AY268050 (mouse) or AY232653 (human), or the complement thereof.

TRAM Nucleic Acid Variants

The invention further encompasses nucleic acid molecules that differ from the nucleotide sequence shown in Genbank accession no. AY268050 (mouse) or AY232653 (human). Such differences can be due to degeneracy of the genetic code (and result in a nucleic acid that encodes the same TRAM polypeptides as those encoded by the nucleotide sequence disclosed herein). In another embodiment, an isolated nucleic acid molecule as described herein has a nucleotide sequence encoding a polypeptide having an amino acid sequence that differs, by at least 1, but less than 5, 10, 20, 50, or 100 amino acid residues, from that shown in Genbank accession no. AY268050 (mouse) or AY232653 (human). If alignment is needed for this comparison the sequences should be aligned for maximum homology. “Looped” out sequences from deletions or insertions, or mismatches, are considered differences.

Nucleic acids can be chosen for having codons, which are preferred, or non-preferred, for a particular expression system. For example, the nucleic acid can be one in which at least one codon, e.g., at least 10%, e.g., 20%, 30%, 40% or more of the codons, has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions, and insertions. Variation can occur in either or both the coding and non-coding regions. The variations can produce both conservative and non-conservative amino acid substitutions (as compared in the encoded product).

In one embodiment, the nucleic acid molecule has a sequence that differs from that of Genbank accession no. AY268050 (mouse) or AY232653 (human) by at least one, but less than 10, 20, 30, or 40 nucleotides, or at least one, but less than 1%, 5%, 10% or 20%, of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in Genbank accession no. AY268050 (mouse) or AY232653 (human) or a fragment of this sequence. Such nucleic acid molecules can readily be identified as being able to hybridize under stringent conditions, to the complement of the nucleotide sequence shown in Genbank accession no. AY268050 (mouse) or AY232653 (human), or a fragment of the sequence. Nucleic acid molecules corresponding to orthologs, homologs, and allelic variants of the TRAM cDNAs described herein can further be isolated by mapping to the same chromosome or locus as the TRAM gene.

Preferred variants include those that are correlated with TRAM signaling.

Allelic variants of TRAM, e.g., human TRAM, include both functional and non-functional polypeptides. Functional allelic variants are naturally occurring amino acid sequence variants of the TRAM polypeptide within a population that maintain the ability to interact with a TRAM-effector and participate in TRAM signaling. Functional allelic variants will typically contain only conservative substitution of one or more (e.g., 5, 10, or 15) amino acids of Genbank accession no. AY268050 (mouse) or AY232653 (human), or substitution, deletion or insertion of non-critical residues in non-critical regions of the polypeptide. Non-functional allelic variants are naturally-occurring amino acid sequence variants of the TRAM, e.g., human TRAM, polypeptide within a population that do not have the ability to interact with a TRAM-effector, localize to the membrane or stimulate an immune response. Non-functional allelic variants will typically contain a non-conservative substitution, a deletion, or insertion, or premature truncation of the amino acid sequence of Genbank accession no. AY268050 (mouse) or AY232653 (human), or a substitution, insertion, or deletion in critical residues or critical regions of the polypeptide.

Moreover, nucleic acid molecules encoding other TRAM family members and, thus, which have a nucleotide sequence that differs from the TRAM sequences of Genbank accession no. AY268050 (mouse) or AY232653 (human), are intended to be within the scope of the invention.

Isolated TRAM Polypeptides

In another aspect, the invention features isolated TRAM polypeptides, or fragments, e.g., biologically active portions, for use as immunogens or antigens to raise or test (or more generally to bind to) anti-TRAM antibodies. TRAM polypeptides can be isolated from cells or tissue sources using standard polypeptide purification techniques. TRAM polypeptides or fragments thereof can be produced by recombinant DNA techniques or synthesized chemically.

The polypeptides described herein include those that arise as a result of alternative transcription events, alternative RNA splicing events, and alternative translational and post-translational events. The polypeptides can be expressed in systems, e.g., cultured cells, which result in substantially the same post-translational modifications present when the polypeptide is expressed in a native cell, or in systems that result in the alteration or omission of post-translational modifications, e.g., glycosylation or cleavage, present when expressed in a native cell.

In some embodiments, a TRAM polypeptide has at least two or more of the following characteristics:
(i) it has the ability to interact with, e.g., bind to or signal through, a TRAM-effector as described herein;
(ii) it has a molecular weight (e.g., a deduced molecular weight, generally ignoring any contribution of post-translational modifications), amino acid composition, and/or other physical characteristic of the polypeptide depicted in Genbank accession no. AY268050 (mouse) or AY232653 (human);
(iii) it has an overall sequence similarity of at least 70%, e.g., at least 80, 90, or 95%, to a polypeptide of Genbank accession no. AY268050 (mouse) or AY232653 (human);
(iv) it can localize to a membrane;
(v) it has a TIR domain that is about 70%, 80%, 90% or 95% identical to the TIR domain of Genbank accession no. AY268050 (mouse, e.g., about amino acids 71-209) or AY232653 (human; e.g., about amino acids 73-232 or 68-186);
(v) it is myristoylated; and
(vi) it activates a NFkB and/or IRF-3 pathway in response to binding of a ligand to TLR4, e.g., LPS or a gram-negative bacteria.

The TRAM polypeptides, or fragments thereof, can differ from the corresponding sequence in Genbank accession no. AY268050 (mouse) or AY232653 (human). In one embodiment, they differ by at least one, but by less than 15, 10, or 5, amino acid residues. In another, they differ from the corresponding sequence in Genbank accession no. AY268050 (mouse) or AY232653 (human) by at least one residue, but less than 20%, 15%, 10% or 5% of the residues in it differ from the corresponding sequence in Genbank accession no. AY268050 (mouse) or AY232653 (human). If this comparison requires alignment, the sequences should be aligned for maximum homology. “Looped” out sequences from deletions or insertions, or mismatches, are considered differences. The differences are, preferably, differences or changes at a non essential residue or a conservative substitution.

Other embodiments include a polypeptide that contains one or more changes in amino acid sequence, e.g., a change in an amino acid residue that is not essential for activity. Such TRAM polypeptides differ in amino acid sequence from Genbank accession no. AY268050 (mouse) or AY232653 (human), yet retain biological activity.

In one embodiment, the polypeptide includes an amino acid sequence at least about 50%, e.g., at least about 55%, 60%, 65%, 70%, 75%, 80%, 85%, 90%, 95%, 98% or more, homologous to Genbank accession no. AY268050 (mouse) or AY232653 (human).

A TRAM polypeptide or fragment is provided that varies from the sequence of the myristoylation site of TRAM by at least one, but by less than 15, 10, or 5, amino acid residues in the polypeptide or fragment. (If this comparison requires alignment the sequences should be aligned for maximum homology. “Looped” out sequences from deletions or insertions, or mismatches, are considered differences.) In some embodiments the difference is at a non essential residue or is a conservative substitution, while in others the difference is at an essential residue or is a non conservative substitution.

In one embodiment, a biologically active portion of a TRAM polypeptide includes a TIR domain and a myristoylation site as described herein. Moreover, other biologically active portions, in which other regions of the polypeptide are deleted, can be prepared by known recombinant techniques and evaluated for one or more of the functional activities of a native TRAM polypeptide.

In some embodiments, the TRAM polypeptide can have an amino acid sequence shown in Genbank accession no. AY268050 (mouse) or AY232653 (human). In other embodiments, the TRAM polypeptide is substantially identical to Genbank accession no. AY268050 (mouse) or AY232653 (human). In yet another embodiment, the TRAM polypeptide is substantially identical to Genbank accession no. AY268050 (mouse) or AY232653 (human) and retains the functional activity of the polypeptide of Genbank accession no. AY268050 (mouse) or AY232653 (human), as described in detail herein.

TRAM Chimeric or Fusion Polypeptides

In another aspect, the invention provides TRAM chimeric or fusion polypeptides. As used herein, a TRAM “chimeric polypeptide” or “fusion polypeptide” includes a TRAM polypeptide linked to a non-TRAM polypeptide. A “non-TRAM polypeptide” refers to a polypeptide having an amino acid sequence corresponding to a polypeptide that is not substantially homologous to the TRAM polypeptide, e.g., a polypeptide that is different from the TRAM polypeptide and that is derived from the same or a different organism. The TRAM polypeptide of the fusion polypeptide can correspond to all or a portion, e.g., a fragment as described herein, of a TRAM amino acid sequence. In one embodiment, a TRAM fusion polypeptide includes at least one (or two) biologically active portion of a TRAM polypeptide. The non-TRAM polypeptide can be fused to the N-terminus or C-terminus of the TRAM polypeptide.

The fusion polypeptide can include a moiety that has a high affinity for a ligand. For example, the fusion polypeptide can be a GST-TRAM fusion polypeptide in which the TRAM sequences are fused to the C-terminus of the GST sequences. Such fusion polypeptides can facilitate the purification of recombinant TRAM. Examples of such fusion polypeptides are provided herein. Alternatively, the fusion polypeptide can be a TRAM polypeptide containing a heterologous signal sequence at its N-terminus. In certain host cells (e.g., mammalian host cells), expression and/or secretion of TRAM can be increased through use of a heterologous signal sequence.

Fusion polypeptides can include all or a part of a serum polypeptide, e.g., an IgG constant region, or human serum albumin.

The TRAM fusion polypeptides described herein can be incorporated into pharmaceutical compositions and administered to a subject in vivo. The TRAM fusion polypeptides can be used to affect the bioavailability of a TRAM substrate. TRAM fusion polypeptides may be useful therapeutically for the treatment of disorders caused by, for example, (i) aberrant modification or mutation of a gene encoding a TRAM polypeptide; (ii) mis-regulation of the TRAM gene; and (iii) aberrant post-translational modification of a TRAM polypeptide.

Moreover, the TRAM-fusion polypeptides described herein can be used as immunogens to produce anti-TRAM antibodies in a subject, to purify TRAM ligands, and in screening assays to identify molecules that inhibit the interaction of TRAM with a TRAM substrate.

Expression vectors are commercially available that already encode a fusion moiety (e.g., a GST polypeptide). A TRAM-encoding nucleic acid can be cloned into such an expression vector so that the fusion moiety is linked in-frame to the TRAM polypeptide.

Screening Assays

The invention also provides methods (also referred to herein as “screening assays”) for identifying modulators, e.g., test compounds or agents (e.g., antibodies, polypeptides, peptides, peptidomimetics, peptoids, small non-nucleic acid organic molecules, small inorganic molecules, oligonucleotides (such as antisense oligonucleotides, ribozymes, or siRNA), or other drugs) that bind to TRAM polypeptides, have a stimulatory or inhibitory effect on, for example, TRAM expression or TRAM activity, or have a stimulatory or inhibitory effect on, for example, the expression or activity of a TRAM substrate or TRAM-effector (a polypeptide that interacts with TRAM as assayed by co-precipitation). TRAM effectors include TLR4, TRIF, Mal, CBP, and p300. Compounds thus identified can be used to modulate the activity of target gene products (e.g., TRAM) in a therapeutic protocol, to elaborate the biological function of the target gene product, or to identify compounds that disrupt normal target gene interactions.

Provided herein are assays for screening candidate or test compounds that are substrates of a TRAM polypeptide or polypeptide or a biologically active portion thereof. In another embodiment, the methods described herein include assays for screening candidate or test compounds that bind to or modulate the activity of a TRAM polypeptide or polypeptide or a biologically active portion thereof.

In one such method of identifying a candidate compound that modulates the interaction between a TRAM and a TRAM-effector, a TRAM polypeptide (e.g., a complete TRAM polypeptide or a portion of a TRAM polypeptide that is required for interaction with the TRAM-effector) and a TRAM-effector are contacted with a test compound and the effect (e.g., increase or decrease) of the test compound on the interaction is assayed. In general, the assay involves detection of the interaction, for example, by labeling one component (e.g., a fusion polypeptide that includes a fluorescent polypeptide and a TRAM-effector or a portion thereof) and using a ligand (such as an antibody) that specifically binds to the second component, e.g., TRAM. The sample containing the two components is contacted with the ligand and the labeled component is detected. The amount of labeled component captured by the ligand indicates the amount of interaction. A test compound can be included in a sample containing the two components and the amount of interaction between the components in the presence of the test compound is compared to the amount of interaction in the absence of the test compound (control). A decrease in the amount of interaction in the presence of the test compound compared to the amount of interaction in the absence of the test compound indicates that the test compound is a candidate compound for decreasing the interaction and is also a candidate compound for decreasing signaling by the components (e.g., signaling by TRAM). Conversely, if the test compound increases the amount of the interaction, e.g., by decreasing the rate at which the two components dissociate from each other, the test compound is a candidate compound for increasing the signaling mediated by the components (e.g., TRAM signaling). Such assays can be cell-free or can be in a cell, e.g., a cell that has been engineered to express one or more of the components.

Another assay method determines whether a test compound can modulate TRAM signaling by providing a test cell that can exhibit TRAM signaling; contacting the test cell with an inducer of TRAM signaling such as TLR4 and a test compound. The amount of expression or activity of TRAM is then determined, and can be compared to a suitable control. For example, the amount or level of TRAM RNA can be determined using hybridization methods known in the art, detection of the amount of TRAM polypeptide present in the cell (e.g., using an antibody that specifically binds TRAM and detecting the bound antibody), or using a cell that is engineered to express a detectable TRAM (e.g., a GFP-TRAM fusion polypeptide). Assays for the ability of a test compound to modulate TRAM expression or activity is then performed by contacting the cell with a test compound and determining the effect of the test compound on TRAM expression or activity (e.g., increasing or decreasing expression or activity).

Another assay relates to a method of modulating the ability of a cell to effect TLR4 signaling. In this method a cell that can undergo TLR4 signaling is contacted with a compound that modulates TRAM expression or activity in an amount sufficient to modulate expression or activity of TRAM. The cell can then be tested for TLR4 signaling, e.g., by monitoring activation of RANTES, IFNβ, or IP-10 activation. Test compounds include antibodies, siRNA, and compounds that affect myristoylation of TRAM, as well as other compounds described herein. Such compounds can decrease TLR4 signaling and therefore decrease the immune response or increase TLR4 signaling, thus increasing the immune response.

Methods of detecting TLR signaling are also included herein. In one such assay, a bone marrow-derived macrophage that expresses a TLR (e.g., TRL-3 or TLR4) is contacted with an inducer of TLR signaling, and secretion of RANTES, activation of IFN-β, or the level of expression of IP10 are detected. An increase in any of these indicates that TLR signaling is increased. Accordingly, this system can be used to assay the test compounds for their ability to modulate TLR signaling.

The test compounds described herein can be obtained using any of the numerous approaches in combinatorial library methods known in the art, including: biological libraries; peptoid libraries (libraries of molecules having the functionalities of peptides, but with a novel, non-peptide backbone that are resistant to enzymatic degradation, but that nevertheless remain bioactive; see, e.g., Zuckermann et al., J. Med. Chem. 37: 2678-85, 1994; spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer, or small molecule libraries of compounds (Lam, K. S., Anticancer Drug Des. 12: 145, 1994).

Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and in Gallop et al., J. Med. Chem. 37: 1233, 1994.

Libraries of compounds may be presented in solution (e.g., Houghten, Biotechniques 13: 412-21, 1994), or on beads (Lam, Nature 354: 82-4, 1994), chips (Fodor, Nature 364: 555-6, 1994), bacteria (Ladner, U.S. Pat. No. 5,223,409), spores (Ladner U.S. Pat. No. '409), plasmids (Cull et al., Proc. Natl. Acad. Sci. USA 89: 1865-69, 1992) or on phage (Scott and Smith, Science 249: 386-90, 1990; Devlin, Science 249: 404-6, 1990; Cwirla et al., Proc. Natl. Acad. Sci. 87: 6378-82, 1990; Felici, J. Mol. Biol. 222: 301-10, 1991; Ladner, supra).

In one embodiment, an assay is a cell-based assay in which a cell that expresses a TRAM polypeptide or biologically active portion thereof is contacted with a test compound, and the ability of the test compound to modulate TRAM activity is determined. The ability of the test compound to modulate TRAM activity can be determined by monitoring, for example, activation of NF-κB, IRF-3, IRF-7, RANTES, and/or IFNα/β. The cell, for example, can be of mammalian origin, e.g., human or murine.

The ability of the test compound to bind to TRAM, or to modulate TRAM binding to a compound such as a TRAM-effector, such as TLR4, TRIF or Mal, can also be evaluated. This can be accomplished, for example, by coupling the compound, e.g., the TRAM-effector, with a radioisotope or enzymatic label such that binding of the compound, e.g., the TRAM-effector, to TRAM can be determined by detecting the labeled compound, e.g., TRAM-effector, in a complex. Alternatively, TRAM could be coupled with a radioisotope or enzymatic label to monitor the ability of a test compound to modulate TRAM binding to a TRAM-effector in a complex. For example, compounds (e.g., TRAM or TRAM-effectors) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product or labeled substrate polypeptides can be produced using recombinant techniques, e.g., to produce a chimeric polypeptide containing sequence from the TRAM substrate and a fluorescent polypeptide such as green fluorescent polypeptide, yellow fluorescent polypeptide, or red fluorescent polypeptide.

The ability of a compound (e.g., a TRAM substrate) to interact with TRAM with or without the labeling of any of the interactants can be evaluated. For example, a microphysiometer can be used to detect the interaction of a compound with TRAM without the labeling of either the compound or TRAM. McConnell, H. M. et al., Science 257: 1906-12, 1992. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and TRAM.

In yet another embodiment, a cell-free assay is provided in which a TRAM polypeptide or biologically active portion thereof is contacted with a test compound and the ability of the test compound to bind to the TRAM polypeptide or biologically active portion thereof is evaluated. Biologically active portions of the TRAM polypeptides to be used in assays of the present invention include fragments that participate in interactions with non-TRAM molecules, e.g., fragments with high surface probability scores.

Soluble and/or membrane-bound forms of isolated polypeptides (e.g., TRAM polypeptides or biologically active portions thereof) can be used in the cell-free assays described herein. When membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene polypeptide and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ polypeptide molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

In another embodiment, determining the ability of the TRAM polypeptide to bind to a target molecule can be accomplished using real-time Biomolecular Interaction Analysis (BIA) (see, e.g., Sjolander, S. and Urbaniczky, C., Anal. Chem. 63: 2338-45, 1991 and Szabo et al., Curr. Opin. Struct. Biol. 5: 699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the TRAM or the test substance is anchored onto a solid phase. The target gene product/test compound complexes anchored on the solid phase can be detected at the end of the reaction. The target gene product can be anchored onto a solid surface, and the test compound, (which is not anchored), can be labeled, either directly or indirectly, with detectable labels discussed herein.

It may be desirable to immobilize either TRAM, an anti-TRAM antibody, or its target molecule to facilitate separation of complexed from uncomplexed forms of one or both of the polypeptides, as well as to accommodate automation of the assay. Binding of a test compound to a TRAM polypeptide, or interaction of a TRAM polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion polypeptide can be provided that adds a domain that allows one or both of the polypeptides to be bound to a matrix. For example, glutathione-S-transferase/TRAM fusion polypeptides or glutathione-S-transferase/target fusion polypeptides can be adsorbed onto glutathione SEPHAROSE™ beads (Sigma Chemical, St. Louis, Mo.) or glutathione derivatized microtiter plates, which are then combined with the test compound or the test compound and either the non-adsorbed target polypeptide or TRAM polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of TRAM binding or activity determined using standard techniques.

Other techniques for immobilizing either a TRAM polypeptide or a target molecule on matrices include using conjugation of biotin and streptavidin. Biotinylated TRAM polypeptide or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with TRAM polypeptide or target molecules but that do not interfere with binding of the TRAM polypeptide to its target molecule. Such antibodies can be derivatized to the wells of the plate, and unbound target or TRAM polypeptide trapped in the wells by antibody conjugation. Methods for detecting such complexes, in addition to those described above for the GST-immobilized complexes, include immunodetection of complexes using antibodies reactive with the TRAM polypeptide or target molecule, as well as enzyme-linked assays that rely on detecting an enzymatic activity associated with the TRAM polypeptide or target molecule.

Alternatively, cell-free assays can be conducted in a liquid phase. In such an assay, the reaction products are separated from unreacted components, by any of a number of known techniques, including but not limited to: differential centrifugation (see, for example, Rivas and Minton, Trends. Biochem. Sci. 18: 284-7, 1993); chromatography (gel filtration chromatography, ion-exchange chromatography); electrophoresis; and immunoprecipitation. Suitable methods are known in the art; see, for example, Ausubel, F. et al., eds. Current Protocols in Molecular Biology, J. Wiley: New York (1999). Resins and chromatographic techniques are known to those in the art (see, e.g., Heegaard, J. Mol. Recognit. 11: 141-8, 1998; Hage and Tweed, J. Chromatogr. B. Biomed. Sci. Appl. 699: 499-525, 1997). Further, fluorescence energy transfer may also be conveniently utilized, as described herein, to detect binding without further purification of the complex from solution.

In a one embodiment, the assay includes contacting the TRAM polypeptide or biologically active portion thereof with a known compound that binds TRAM to form an assay mixture, contacting the assay mixture with a test compound, and determining the ability of the test compound to interact with a TRAM polypeptide, wherein determining the ability of the test compound to interact with a TRAM polypeptide includes determining the ability of the test compound to preferentially bind to TRAM or biologically active portion thereof, or to modulate the activity of a target molecule, as compared to the known compound.

The target gene products described herein can, in vivo, interact with one or more cellular or extracellular macromolecules, such as polypeptides. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The target genes/products for use in this embodiment are generally TRAM. In an alternative embodiment, the invention provides methods for determining the ability of the test compound to modulate the activity of a TRAM polypeptide through modulation of the activity of a downstream effector of a TRAM target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. To test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

To conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit the formation of complexes or that disrupt preformed complexes can be identified.

In an alternate embodiment, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the TRAM polypeptides can be used as “bait polypeptides” in a two-hybrid assay or three-hybrid assay (see, e.g., U.S. Pat. No. 5,283,317; Zervos et al., Cell 72: 223-32, 1993; Madura et al., J. Biol. Chem. 268: 12046-54, 1993; Bartel et al., Biotechniques 14: 920-4, 1993; Iwabuchi et al., Oncogene 8: 1693-96, 1993; and Brent WO94/10300), to identify other polypeptides that bind to or interact with TRAM (“TRAM-binding polypeptides” or “TRAM-bp”) and are involved in TRAM activity. Such TRAM-bps can be activators or inhibitors of signals by the TRAM polypeptides or TRAM targets as, for example, downstream elements of a TRAM-mediated signaling pathway.

The two-hybrid system is based on the modular nature of most transcription factors, which consist of separable DNA-binding and activation domains. Briefly, the assay utilizes two different DNA constructs. In one construct, the gene that codes for a TRAM polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor (alternatively the TRAM polypeptide can be fused to the activator domain). If the “bait” and the “prey” polypeptides are able to interact, in vivo, forming a TRAM-dependent complex, the DNA-binding and activation domains of the transcription factor are brought into close proximity. This proximity allows transcription of a reporter gene (e.g., lacZ) that is operably linked to a transcriptional regulatory site responsive to the transcription factor. Expression of the reporter gene can be detected and cell colonies containing the functional transcription factor can be isolated and used to obtain the cloned gene that encodes the polypeptide that interacts with the TRAM polypeptide.

In another embodiment, modulators of TRAM expression are identified. For example, a cell or cell free mixture is contacted with a test compound and the expression of TRAM mRNA or polypeptide evaluated relative to the level of expression of TRAM mRNA or polypeptide in the absence of the test compound. When expression of TRAM mRNA or polypeptide is greater in the presence of the test compound than in its absence, the test compound is identified as a stimulator of TRAM mRNA or polypeptide expression. Alternatively, when expression of TRAM mRNA or polypeptide is less (statistically significantly less) in the presence of the test compound than in its absence, the test compound is identified as an inhibitor of TRAM mRNA or polypeptide expression. The level of TRAM mRNA or polypeptide expression can be determined by methods described herein for detecting TRAM mRNA or polypeptide.

In another aspect, the invention pertains to a combination of two or more of the assays described herein. For example, a modulating agent can be identified using a cell-based or a cell free assay, and the ability of the agent to modulate the activity of a TRAM polypeptide can be confirmed in vivo, e.g., in an animal such as a murine model of infection or allergic inflammation.

This invention further pertains to novel agents identified by the above-described screening assays. Accordingly, it is within the scope of this invention to further use an agent identified as described herein (e.g., a TRAM modulating agent, an antisense TRAM nucleic acid molecule, a TRAM-specific antibody, or a TRAM-binding partner) in an appropriate animal model to determine the efficacy, toxicity, side effects, or mechanism of action, of treatment with such an agent. Furthermore, novel agents identified by the above-described screening assays can be used for treatments as described herein.

Animal models, e.g., of viral infection, are known in the art and are described herein. Such models can also be used in assay methods as described herein to identify compounds that modulate the expression or activity of TRAM. Such models can also be used to determine the effects of such compounds on, e.g., an inflammatory response, in vivo cytotoxicity, modulation or viral load, severity of infection or duration of infection. Methods of identifying such conditions are known in the art.

Compounds that can be used in the assays described herein and that can be useful as pharmaceutical compositions also include siRNA, ribozymes, and antisense oligonucleotides. Methods of making such compounds are known in the art and are described below.

RNA Interference

RNAi is an efficient process whereby double-stranded RNA (dsRNA, also referred to herein as siRNAs or ds-siRNAs, for small interfering or double-stranded small interfering RNAs,) induces the sequence-specific degradation of homologous mRNA in animals and plant cells (Hutvagner and Zamore, Curr. Opin. Genet. Dev. 12: 225-32, 2002; Sharp, Genes Dev. 15: 485-90, 2001). In mammalian cells, RNAi can be triggered by 21-nucleotide (nt) duplexes of small interfering RNA (siRNA) (Chiu et al., Mol. Cell. 10: 549-561, 2002; Elbashir et al., Nature 411: 494-98, 2001, or by micro-RNAs (mRNA), functional small-hairpin RNA (shRNA), or other dsRNAs that are expressed in vivo using DNA templates with RNA polymerase III promoters (Zeng et al., Mol. Cell 9: 1327-33, 2002; Paddison et al., Genes Dev. 16: 948-58, 2002; Lee et al., Nature Biotechnol. 20: 500-5, 2002; Paul et al., Nature Biotechnol. 20: 505-8, 2002; Tuschl, Nature Biotechnol. 20: 440-48, 2002; Yu et al., Proc. Natl. Acad. Sci. USA 99(9): 6047 52, 2002; McManus et al., RNA 8: 842-50, 2002; Sui et al., Proc. Natl. Acad. Sci. USA 99(6): 5515-20, 2002).

Accordingly, the invention includes such molecules that are targeted to a TRAM RNA. Molecules that can decrease the amount of TRAM RNA are useful for decreasing or preventing undesirable immune system activation, e.g., an undesirable inflammatory response. Molecules that can decrease the amount of TRAM RNA are useful for increasing an immune response, e.g., to increase the efficacy of a vaccine.

siRNA Molecules

The nucleic acid molecules or constructs described herein include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules described herein can be chemically synthesized, or can transcribed be in vitro from a DNA template, or in vivo from, e.g., shRNA. The dsRNA molecules can be designed using any method known in the art. For example, siRNAs or pools thereof can be obtained from commercial vendors including Ambion and Dharmacon. InvivoGen provides a ready-made set of TRAM psiRNAs, plasmids useful for expressing siRNAs targeting mouse and human TRAM (InvivoGen, San Diego, Calif.).

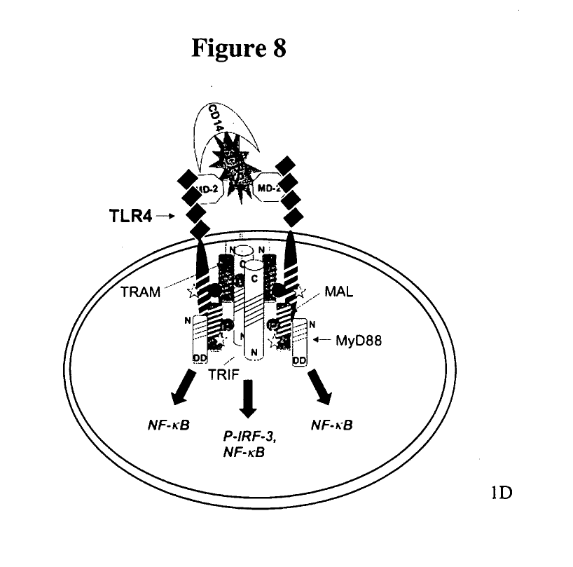

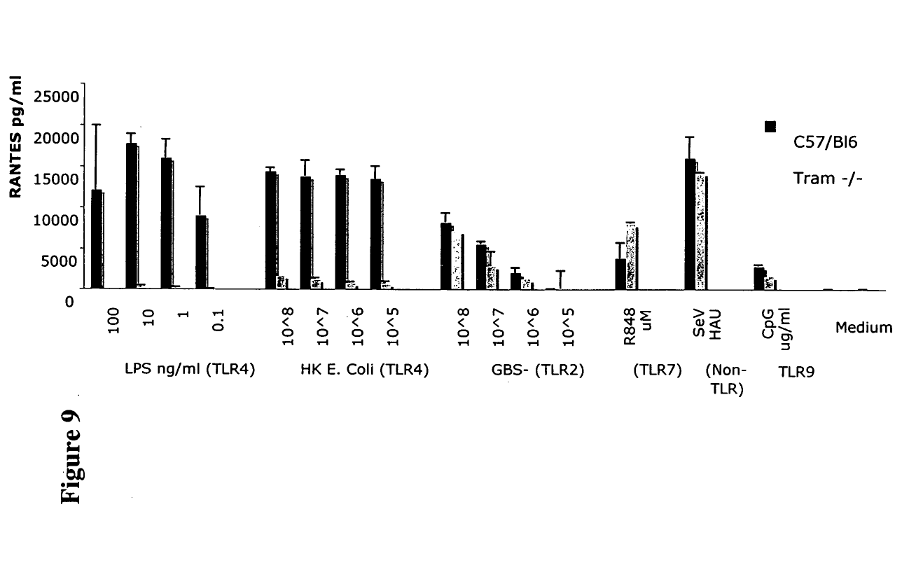

A number of algorithms are known, including the following protocol:
1. Beginning with the AUG start codon, look for AA dinucleotide sequences; each AA and the 3′ adjacent 16 or more nucleotides are potential siRNA targets (see FIGS. 8 and 9). siRNAs taken from the 5′ untranslated regions (UTRs) and regions near the start codon (within about 75 bases or so) may be less useful as they may be richer in regulatory polypeptide binding sites, and UTR-binding polypeptides and/or translation initiation complexes may interfere with binding of the siRNP or RISC endonuclease complex. Thus, in one embodiment, the nucleic acid molecules are selected from a region of the cDNA sequence beginning 50 to 100 nt downstream of the start codon. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TT. The overhanging nucleotides can be either RNA or DNA.
2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at www.ncbi.nlm.nih.gov/BLAST.
3. Select one or more sequences that meet your criteria for evaluation.

Further general information about the design and use of siRNA can be found in “The siRNA User Guide,” available at mpibpc.gwdg.de/abteilungen/100/105/sirna.html.

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The nucleic acid compositions described herein include both siRNA and crosslinked siRNA derivatives. Crosslinking can be employed to alter the pharmacokinetics of the composition, for example, to increase half-life in the body. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions described herein can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al. (Drug Deliv. Rev. 47(1): 99-112, 2002; describing nucleic acids loaded to polyalkylcyanoacrylate (PACA) nanoparticles); Fattal et al. (J. Control Release 53(1-3): 137-43, 1998, which describes nucleic acids bound to nanoparticles); Schwab et al. (Ann. Oncol. 5 Suppl. 4: 55-8, 1994; which describes nucleic acids linked to intercalating agents, hydrophobic groups, polycations, or PACA nanoparticles); and Godard et al. (Eur. J. Biochem. 232(2): 404-10, 1995; describes nucleic acids linked to nanoparticles).

The nucleic acid molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

siRNA Delivery for Longer-Term Expression

Synthetic siRNAs can be delivered into cells by cationic liposome transfection and electroporation. However, these exogenous siRNA show only short term persistence of the silencing effect (generally about 4-5 days). Several strategies for expressing siRNA duplexes within cells from recombinant DNA constructs allow longer-term target gene suppression in cells, including mammalian Pol III promoter systems (e.g., H1 or U6/snRNA promoter systems (Tuschl, Nature Biotechnol. 20: 440-48, 2002) capable of expressing functional double-stranded siRNAs; (Bagella et al., J. Cell. Physiol. 177: 206-13, 1998; Lee et al., Nature Biotechnol. 20: 500-5, 2002; Miyagishi et al., Nucleic Acids Res Suppl. 2002(2): 113-4, 2002; Paul et al., Nature Biotechnol. 20: 505-8, 2002; Yu et al., Proc. Natl. Acad. Sci. USA 99(9): 6047-52, 2002; Sui et al., Proc. Natl. Acad. Sci. USA 99(6): 5515-20, 2002). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al., J. Cell. Physiol. 177: 206-13, 1998; Lee et al., Nature Biotechnol. 20: 500-5, 2002; Miyagishi et al., 2002, supra; Paul et al., Nature Biotechnol. 20: 505-8, 2002; Yu et al., Proc. Natl. Acad. Sci. USA 99(9): 6047-52, 2002; Sui et al., Proc. Natl. Acad. Sci. USA 99(6): 5515-20, 2002). Constructs containing siRNA sequence under the control of T7 promoter also make functional siRNAs when cotransfected into the cells with a vector expression T7 RNA polymerase (Jacque et al., Nature 418(6896): 435-8, 2002). Modified siRNAs can also be used, see, e.g., Layzer et al., RNA 10(5): 766-71, 2004.

Animal cells express a range of noncoding RNAs of approximately 22 nucleotides termed micro RNA (mRNAs) and can regulate gene expression at the post transcriptional or translational level during animal development. One common feature of mRNAs is that they are all excised from an approximately 70 nucleotide precursor RNA stem-loop, probably by Dicer, an RNase III-type enzyme, or a homolog thereof. By substituting the stem sequences of the mRNA precursor with mRNA sequence complementary to the target mRNA, a vector construct that expresses the novel mRNA can be used to produce siRNAs to initiate RNAi against specific mRNA targets in mammalian cells (Zeng et al., Mol. Cell 9: 1327-33, 2002). When expressed by DNA vectors containing polymerase III promoters, micro-RNA designed hairpins can silence gene expression (McManus et al., RNA 8: 842-50, 2002). Viral-mediated delivery mechanisms can also be used to induce specific silencing of targeted genes through expression of siRNA, for example, by generating recombinant adenoviruses harboring siRNA under RNA Pol II promoter transcription control (Xia et al., Nat Biotechnol. 20(10): 1006-10, 2002). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. In an animal model, whole-embryo electroporation can efficiently deliver synthetic siRNA into post-implantation mouse embryos (Calegari et al., Proc. Natl. Acad. Sci. USA 99(22): 14236-40, 2002). In adult mice, efficient delivery of siRNA can be accomplished by “high-pressure” delivery technique, a rapid injection (within 5 seconds) of a large volume of siRNA containing solution into animal via the tail vein (McCaffrey et al., Nature 418(6893): 38-9., 2002; Lewis, Nature Genetics 32: 107-8, 2002). Nanoparticles and liposomes can also be used to deliver siRNA into animals.

Uses of Engineered RNA Precursors to Induce RNAi

Engineered RNA precursors, introduced into cells or whole organisms as described herein, will lead to the production of a desired siRNA molecule. Such an siRNA molecule will then associate with endogenous polypeptide components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the polypeptide encoded by that mRNA in the cell or organism.

Antisense

An “antisense” nucleic acid can include a nucleotide sequence that is complementary to a “sense” nucleic acid encoding a polypeptide, e.g., complementary to the coding strand of a double-stranded cDNA molecule or complementary to a target mRNA sequence, e.g., a TRAM mRNA sequence.

An antisense nucleic acid can be designed such that it is complementary to the entire coding region of a target mRNA, e.g., a TRAM mRNA, but can also be an oligonucleotide that is antisense to only a portion of the coding or noncoding region (e.g., the 5′ or 3′ untranslated regions) of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

Based upon the sequences disclosed herein, one of skill in the art can easily choose and synthesize any of a number of appropriate antisense molecules for use in accordance with the present invention. For example, a “gene walk” comprising a series of oligonucleotides of 15-30 or more nucleotides spanning the length of a TRAM nucleic acid can be prepared, followed by testing for inhibition of TRAM expression. Optionally, gaps of 5-10 nucleotides can be left between the oligonucleotides to reduce the number of oligonucleotides synthesized and tested.

An antisense nucleic acid described herein can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection). The antisense nucleic acids can be morpholino oligos.

The antisense nucleic acid molecules described herein are typically administered to a subject (e.g., by direct injection at a tissue site), or generated in situ such that they hybridize with or bind to cellular mRNA and/or genomic DNA encoding a target polypeptide to thereby inhibit expression of the polypeptide, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecules described herein are α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15: 6625-41, 1987). The antisense nucleic acid molecule can also comprise a 2′-o-methylribonucleotide (Inoue et al., Nucleic Acids Res. 15: 6131-48, 1987) or a chimeric RNA-DNA analogue (Inoue et al. FEBS Lett., 215: 327-30, 1987).

Gene expression of a target polypeptide (e.g., TRAM) can be inhibited by targeting nucleotide sequences complementary to the regulatory region of a target gene (e.g., promoters and/or enhancers) to form triple helical structures that prevent transcription of the target gene in a cells. See generally, Helene, C., Anticancer Drug Des. 6: 569-84, 1991; Helene, C. Ann. N.Y. Acad. Sci. 660: 27-36, 1992; and Maher, Bioassays 14: 807-15, 1992. The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Antisense sequences that decrease expression of TRAM are useful for, e.g., decreasing an immune response.

Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a target-encoding nucleic acid (e.g. TRAM mRNA) can include one or more sequences complementary to the nucleotide sequence of the target cDNA, and a sequence having known catalytic sequence responsible for mRNA cleavage (see U.S. Pat. No. 5,093,246 or Haselhoff and Gerlach, Nature 334: 585-91, 1988). For example, a derivative of a Tetrahymena L-19 IVS RNA can be constructed in which the nucleotide sequence of the active site is complementary to the nucleotide sequence to be cleaved in a target-encoding mRNA. See, e.g., Cech et al. U.S. Pat. No. 4,987,071; and Cech et al. U.S. Pat. No. 5,116,742. Alternatively, a target mRNA can be used to select a catalytic RNA having a specific ribonuclease activity from a pool of RNA molecules. See, e.g., Bartel, D. and Szostak, J. W., Science 261: 1411-18, 1993.

Ribozymes that cleave TRAM are useful, e.g., for inhibiting an immune response.

Cells for Use in Assays Described Herein

Cells useful for the assays described herein include, but are not limited to, cells that inherently express one or more polypeptides such as TRAM, TRIF, TLR3, or TLR4, and cells that have been engineered to express one or more such polypeptides. Engineered cells can be transiently transfected with a an expression vector or can stably express a recombinant polypeptide. Cells that can be used include cells that naturally express TLRs such as myeloid dendritic cells (DCs), which express TLR4 and other TLRs. Examples of useful cell lines are discussed in the Examples and include HELA and HEK293 cells. In addition, cells in which one or more of the polypeptides, e.g., TRAM, TRIF, Mal, MyD88, or TLR4, have been knocked out permanently or transiently can also be used.

Correlating Information

Methods for correlating information about a test compound are also included herein. Correlating means identifying a test compound that interacts with TRAM or modulates TRAM expression, levels, or activity as an compound that can modulate immune system activity. The correlating step can include, e.g., generating or providing a record, e.g., a print or computer readable record, such as a laboratory record, electronic mail, or dataset, identifying a test compound that interacts with TRAM and modulates TRAM activity, or a test compound that modulates TRAM expression or levels as a compound that can modulate immune system activity. The record can include other information, such as a specific test compound identifier, a date, an operator of the method, or information about the source, structure, method of purification or biological activity of the test compound. The record or information derived from the record can be used, e.g., to identify the test compound as a compound or candidate compound (e.g., a lead compound) for pharmaceutical or therapeutic use. The identified compound can be identified as an agent or a potential agent for treatment of diabetic nephropathy. Agents, e.g., compounds, identified by this method can be used, e.g., in the treatment (or development of treatments) for immune system related disorders or for increasing the activity of the immune system (e.g., as adjuvants for vaccine administration).

Antibodies

Anti-TRAM antibodies (TRAM antibody) are also included herein. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin.

Human TRAM is a 235 amino acid polypeptide with a short N-terminal myristoylation site-containing domain and a COOH-terminal Toll-Interleukin-1-Resistance (TIR) domain. Unlike MyD88, it lacks a death domain. TRAM is similar to the other adapter molecules, particularly in its TIR domain. However, the N-terminal 66 amino acids of human/and or mouse TRAM are unique to TRAM. An additional region unique to TRAM is the extreme C-terminal portion of the molecule, just C-terminal to the TIR domain, which it does not share with related molecules such as TRIF, MyD88 or Mal. Although antibodies generated to other regions of TRAM are useful (e.g., antibodies that specifically bind to the TIR domain), antibodies that specifically bind to the N-terminus or a portion thereof are useful, e.g., for specifically identifying TRAM.

The antibody can be, e.g., polyclonal, monoclonal, monospecific, or recombinant, e.g., a chimeric or humanized, fully human, non-human, e.g., murine, or single chain antibody. In some embodiments, the antibody has effector function and can fix complement. The antibody can be coupled to a toxin or imaging agent.

A full-length TRAM polypeptide or antigenic peptide fragment of TRAM can be used as an immunogen, or can be used to identify anti-TRAM antibodies made with other immunogens, e.g., cells, membrane preparations, and the like. The antigenic peptide of TRAM should include at least 8 amino acid residues of the amino acid sequence shown in Genbank Accession No. AY268050 and encompasses an epitope of TRAM. Generally, the antigenic peptide includes at least 10 amino acid residues, for example, at least 15 amino acid residues, at least 20 amino acid residues, and at least 30 amino acid residues.

Fragments of TRAM that include the myristoylation site (e.g., amino acids 2-18 of human or murine TRAM) or an antigenic fragment thereof, or the TIR domain (e.g., the 66-235 amino acids at the COOH terminus of TRAM or a fragment thereof) can be used e.g., as immunogens or to characterize the specificity of an antibody.

Antibodies reactive with, or specific for, any of these regions, or other regions or domains described herein are provided.

In general, epitopes encompassed by the antigenic peptide are regions of TRAM are located on the surface of the polypeptide, e.g., hydrophilic regions, as well as regions with high antigenicity. For example, an Emini surface probability analysis of the human TRAM polypeptide sequence can be used to indicate the regions that have a particularly high probability of being localized to the surface of the TRAM polypeptide and are thus likely to constitute surface residues useful for targeting antibody production.

In general, the antibody binds an epitope on any domain or region on TRAM polypeptides described herein.

Chimeric, humanized, or completely human antibodies are desirable for applications that include repeated administration, e.g., therapeutic treatment (and some diagnostic applications) of human patients.

The anti-TRAM antibody can be a single chain antibody. A single-chain antibody (scFV) can be engineered (see, for example, Colcher et al., Ann. N.Y. Acad. Sci. 880: 263-80, 1999; and Reiter, Clin. Cancer. Res. 2: 245-52, 1996). The single chain antibody can be dimerized or multimerized to generate multivalent antibodies having specificities for different epitopes of the same target TRAM polypeptide.

In a some cases, the antibody has reduced or no ability to bind an Fc receptor. For example., it is a isotype or subtype, fragment or other mutant, which does not support binding to an Fc receptor, e.g., it has a mutagenized or deleted Fc receptor binding region.

An anti-TRAM antibody (e.g., monoclonal antibody) can be used to isolate TRAM by standard techniques, such as affinity chromatography or immunoprecipitation. Moreover, an anti-TRAM antibody can be used to detect TRAM polypeptide (e.g., in a cellular lysate or cell supernatant) in order to evaluate the abundance and pattern of expression of the polypeptide, for example, in screening assays for identifying modulators of TRAM expression or activity. Anti-TRAM antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Methods of Treatment

Provided herein are both prophylactic and therapeutic methods of treating a subject at risk of (or susceptible to) a disorder or having a disorder associated with TRAM and/or TLR4 signaling, e.g., a bacterial or viral infection, or a disorder associated with undesirable inflammation such as rheumatoid arthritis. As used herein, the term “treatment” is defined as the application or administration of a therapeutic agent to a subject, e.g., a patient who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes the compounds described herein and includes, but is not limited to, small molecules, peptides, peptidomimetics, antibodies, ribozymes, siRNA, and antisense oligonucleotides.

It is possible that some disorders involving TRAM signaling can be caused, at least in part, by an abnormal level of gene product (e.g., TRAM), or by the presence of a gene product exhibiting abnormal activity. As such, the reduction in the level and/or activity of such gene products would bring about the amelioration of disorder symptoms.

The compounds that modulate that are identified as described herein can be used to treat and/or diagnose a variety of immune disorders, particularly those involving activation of the innate immune system. Examples of such disorders or diseases include, but are not limited to, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

In general, induction or enhancement of TRAM signaling is useful for treating infection by a virus or other pathogen that can induce TRAM signaling, e.g., to enhance the immune response to the virus or pathogen. Alternatively, when the immune response is undesirably robust, e.g., in the case of inflammation, inhibition of TRAM signaling is useful.

As discussed herein, successful treatment of disorders associated with TRAM signaling can be brought about by techniques that serve to modulate the expression or activity of TRAM. For example, a compound, e.g., an agent identified using an assay described herein, that exhibits negative modulatory activity of TRAM, can be used to prevent and/or ameliorate symptoms of inflammatory disorders. Such molecules can include, but are not limited to peptides, phosphopeptides, small non-nucleic acid organic molecules, (e.g., anti-sense oligonucleotides, morpholino oligos, ribozymes, or siRNA) or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof).

Antisense and ribozyme molecules that inhibit expression of a target gene (e.g., TRAM) can also be used in accordance with the invention to reduce the level of target gene expression, thus effectively reducing the level of target gene activity. Still further, triple helix molecules can be utilized in reducing the level of target gene activity. Methods of using such molecules are known in the art.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target polypeptides exhibiting normal target gene activity can be introduced into cells via gene therapy methods.

Another method by which nucleic acid molecules may be utilized in treating or preventing a disease characterized by target expression is through the use of aptamer molecules specific for a target polypeptide. Aptamers are nucleic acid molecules having a tertiary structure that permits them to specifically bind to polypeptide ligands (see, e.g., Osborne et al. Curr. Opin. Chem. Biol. 1: 5-9, 1997; and Patel, Curr. Opin. Chem. Biol. 1: 32-46, 1997). Since nucleic acid molecules may in many cases be more conveniently introduced into target cells than therapeutic polypeptide molecules may be, aptamers offer a method by which a target polypeptide activity may be specifically decreased without the introduction of drugs or other molecules which may have pluripotent effects.

Antibodies can be generated that are both specific for a target and that reduce target activity (e.g., by interfering with the ability of TRAM to participate in TRAM signaling). Such antibodies may, therefore, be administered in instances whereby negative modulatory techniques are appropriate for the treatment of disorders related to TRAM signaling. For example, such compounds are useful when it is desirable to decrease TRAM signaling.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., Proc. Natl. Acad. Sci. USA 90: 7889-93, 1993).

The identified compounds that inhibit target gene expression, synthesis and/or activity can be administered to a patient at therapeutically effective doses to prevent, treat or ameliorate disorders associated with TRAM signaling. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound that is able to modulate TRAM signaling is used as a template, or “imprinting molecule,” to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell et al., Current Opinion in Biotechnology 7: 89-94, 1996 and in Shea, Trends in Polymer Science 2: 166-73, 1994. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, et al. Nature 361: 645-47, 1993. Through the use of isotope-labeling, the “free” concentration of compound that modulates TRAM signaling can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz, et al. Analytical Chemistry 67: 2142-44, 1995.

Another method described herein pertains to methods of modulating the expression or activity of a target (e.g., TRAM) for therapeutic purposes. Accordingly, in an exemplary embodiment, the modulatory methods described herein involve contacting a cell with a target molecule (e.g., a TRAM or biologically active fragment thereof) or agent that modulates one or more of the associated activities associated with the target.

In one embodiment, the agent stimulates the expression or activity of a target. For example, the agent can stimulate the expression or activity of TRAM, thus enhancing TRAM signaling and the immune response, e.g., to increase the efficacy of a vaccine. In another embodiment, the agent inhibits one or more activities associated with TRAM signaling as described herein. Examples of such inhibitory agents (e.g., agents that inhibit TRAM signaling) include antisense nucleic acid molecules, antibodies, and inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by an aberrant or unwanted immune or inflammatory response, or in conditions in which it is desirable to increase an immune response or an inflammatory response (e.g., during viral infection). In one embodiment, the method involves administering an agent (e.g., an agent identified by a screening assay described herein), or combination of agents that modulates (e.g., up regulates or down regulates) a target (e.g., TRAM) expression or activity. In another embodiment, the method involves administering a target nucleic acid molecule as therapy to compensate for reduced, aberrant, or unwanted target expression or activity.

Stimulation of TRAM activity is desirable in situations in which TRAM is abnormally downregulated and/or in which increased TRAM activity is likely to have a beneficial effect e.g., for eliciting a robust response to a vaccine, e.g., to increase an antiviral response. Likewise, inhibition of TRAM activity is desirable in situations in which TRAM is abnormally upregulated and/or in which decreased TRAM activity is likely to have a beneficial effect, for example when it is desirable to decrease an inflammatory response such as an inflammatory response caused by vaccination or an inflammatory disorder.

Pharmaceutical Compositions

Compounds that modulate the expression or activity of TRAM (e.g., during viral infection or vaccination) or other compounds identified as described herein can be incorporated into pharmaceutical compositions. Such compositions typically include the compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral (e.g., intravenous, intradermal, subcutaneous), oral, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds generally lies within a range of circulating concentrations that include the ED50 with little or no toxicity. The dosage may vary within this range depending upon the dosage form employed and the route of administration utilized. For any compound used in the methods described herein, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of polypeptide or polypeptide (i.e., an effective dosage) ranges from about 0.001 to 30 mg/kg body weight, about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, or 5 to 6 mg/kg body weight. The polypeptide or polypeptide can be administered one time per week for between about 1 to 10 weeks, between 2 to 8 weeks, between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a polypeptide, polypeptide, or antibody can include a single treatment or can include a series of treatments.

For antibodies, the dosage is generally about 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. (J. Acq. Imm. Def. Syn. Hum. Retrovirol. 14: 193, 1997).

The present invention encompasses agents that modulate expression or activity. An agent can, for example, be a small molecule. Such small molecules include, but are not limited to, peptides, peptidomimetics (e.g., peptoids), amino acids, amino acid analogs, polynucleotides (e.g., siRNA or antisense RNA), polynucleotide analogs, nucleotides, nucleotide analogs, non-nucleic acid organic compounds or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, sex, and diet of the subject, the time of administration, the route of administration, the rate of excretion, any drug combination, and the degree of expression or activity to be modulated.

A nucleic acid molecule that modulates TRAM expression or activity or exhibits one of the other desired activities described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., Proc. Natl. Acad. Sci. USA 91: 3054-57, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

A compound as described herein can be used for the preparation of a medicament for use in any of the methods of treatment described herein.

Some embodiments of the invention are illustrated by the following Examples, which are not to be considered limiting.

### EXAMPLES

Materials and Methods

Reagents

An IRF-3-ΔN, Gal4-IRF-3 and Gal4-luciferase reporter gene were from T. Fujita (Tokyo, Japan;) Shinobu et al., FEBS Lett 517: 251-256, 2002. IKKε-k38a and TBK1-k38a were as described in (Peters et al., Mol. Cell 5: 513-22, 2000; Fitzgerald et al., Nat. Immunol. 4: 491-496, 2003). IRF-7, IRF-7ΔN and Gal4-IRF-7 were from P. Pitha (Baltimore, Md.) (Au et al., J. Biol. Chem. 273: 29210-217, 1998). The RANTES-reporter construct was as described in Lin et al., Mol. Cell Biol. 19: 959-966, 1999. The IP-10 reporter construct was from A. Luster (Massachusetts General Hospital, Boston, Mass.). The NF-κB-luciferase construct (Fitzgerald et al., Nature 413: 78-83, 2001, pEF-Bos-Flag Mal and Flag-TRIF were as described in Fitzgerald et al., Nat. Immunol. 4: 491-96, 2003. The plasmids pEF-Bos-Flag-TRAM, TRAM-CFP, TRIF-CFP, and Mal-CFP were generated by PCR cloning from a human PBMC cDNA library. pEF-Bos-TRAM-TIR (amino acids 63-235 of Genbank Accession No. AY232653, The mouse Genbank submission is AY268050, pEF-Bos-TRAM-C117H, TRAM-P116H, and TRIF-β434H were generated using the Quick-Change site-directed mutagenesis kit (Stratagene, La Jolla, Calif.). Polyclonal antibodies to IRF-3 were from Zymed (San Fransisco, Calif.) and CBP antibodies were obtained from Santa Cruz (Santa Cruz, Calif.). PCMV-TRIFΔNΔC, and MyD88-deficient mice were from S. Akira (Osaka, Japan) (Adachi et al., Immunity 9(1): 143-50, 1998) MyD88 knockout mice used were backbred onto a C57BL6 background for five generations. LPS derived from Escherichia coli strain 011:B4 was purchased from Sigma, dissolved in deoxycholate and re-extracted by phenol:chloroform as described in (Hirschfeld et al., J. Immunol. 165: 618-22, 2000). Poly IC was obtained from Amersham Pharmacia (Piscataway, N.J.).

Stable Cell Lines

Clonal stable cell lines were engineered by transfecting HEK293 cells with chimeric fluorescent polypeptide TLR constructs as described in (Latz et al., J. Biol. Chem. 277: 47834-43, 2002). A HEK293 cell line stably expressing both TLR4 and MD-2 was generated using retroviral transduction of HEK/TLR4 cells with a retrovirus encoding human MD2 as described in (Visintin et al., Proc. Natl. Acad. Sci. USA 98: 12156-161, 2001). HEK/TLR3 and HEK/IRF-3-GFP were prepared as described in (Fitzgerald et al., Nat. Immunol. 4: 491-96, 2003) and U373/CD14 cells were prepared as described in (Lien et al., J. Biol. Chem. 276: 1873-80, 2001).

Electrophoretic Mobility Shift Assays

Bone marrow derived macrophages were cultured from C57B16 mice or age and sex-matched MyD88−/− mice for eight days in M-CSF (10 ng/ml). Nuclear extracts from 5×10⁵ cells were purified after LPS (10 ng/ml), Malp-2 (1 nM) or Poly IC (50 μg/ml) stimulation for the times indicated. The extracts were incubated with a specific probe for the ISRE consensus sequence (Promega, Madison, Wis.), electrophoresed, and visualized by autoradiography (Fitzgerald et al., J Immunol 164: 2053-2063, 2000). Supershift analysis was performed with antibodies to mouse IRF-3, p65, or IgG control.

ELISA

Macrophages (5×10⁴ cells per well) were seeded into 96-well plates for 24 hours prior to stimulation with LPS, poly I:C, or medium for 12 hours. Cell culture supernatants were removed and analyzed for the presence of RANTES, IP-10, or TNFα by ELISA (R&D Systems).

Transfection Assays

Cells were seeded into 96-well plates at a density of 1.5×10⁴ cells per well and transfected 24 hours later with 40 ng of the indicated luciferase reporter genes using Genejuice (Novagen). The thymidine kinase Renilla-luciferase reporter gene (Promega) (40 ng/well) was cotransfected as a marker for normalization of data for transfection efficiency. Cell lysates were prepared and reporter gene activity was measured using the Dual Luciferase Assay System (Promega). Data were expressed as the mean relative stimulation±S.D. Experiments were generally performed a minimum of three times.

Immunofluorescence and Confocal Microscopy

A HEK293-IRF-3-GFP stable cell line was transiently transfected with Flag-tagged constructs as indicated. After allowing two days for polypeptide expression to occur, the transfected cells were fixed, permeabilized, and stained with Cy3-conjugated anti-Flag antibody (clone M2, Sigma-Aldrich). DRAQ5 was added to counterstain nuclei. Cells were imaged by confocal microscopy using a Leica TCS SP2 AOBS microscope.

RNA Interference

siRNA duplexes targeting the coding region of TRAM and Lamin A/C were from Dharmacon (Lafayette, Colo.), TRAM-siRNA sequences: GGAAGAAAGTCGTGGATT (SEQ ID NO:1) (product #: D-004334-01™); and Lamin A/C: CTGGACTTCCAGAAGAACA (SEQ ID NO:2). siRNA duplexes targeting the 3′ UTR of TRIF were as described in (Oshiumi et al., Nat. Immunol. 4(2): 161-7, 2003). To determine the efficiency of gene silencing, 293T cells (24 well plates, 4×10⁴ cells/well) were transfected with 0.5 μg of plasmids encoding TRAM-CFP, TRIF-CFP, or Mal-CFP expression vectors. These cells were co-transfected with TRAM or Lamin A/C siRNA duplexes (50 nM) using Mirus TransIT® TKO and TransIT-LT1® transfection reagents in a combination protocol according to the manufacturer's recommendations (Mirus, Madison, Wis.). CFP fluorescence was quantified by flow cytometry (Becton Dickinson, LSR) 24 hours later. For reporter assays, U373/CD14 cells or TLR3-expressing HEK293 cells (4×10⁴ cells/well) were transfected with 0.5 μg of the RANTES reporter gene and 0.25 μg of a thymidine kinase-renilla reporter gene and cotransfected with 50 nM of siRNA targeting vectors as described supra in 24-well tissue culture dishes. Thirty-six hours following transfection, cells were stimulated with LPS or dsRNA for approximately 8 hours before luciferase activity was measured.

Co-Immunoprecipitation

293T cells or TLR-expressing cells (10 cm plates) were transfected using GeneJuice (Novagen) with 4 μg of the indicated plasmids. One to two days after transfection, the cells were lysed in 1 ml of lysis buffer (20 mM Tris-HCl, 2 mM EDTA, 137 mM NaCl, 0.5% Triton X-100, 10% glycerol with protease inhibitors). Polyclonal anti-GFP (Molecular Probes), anti-IRF-3 or anti-CBP antibodies were incubated overnight with the cell lysates in Polypeptide A Sepharose. The immune complexes were precipitated and subjected to 4-15% SDS-PAGE and immunoblotted for FLAG- or CFP/YFP-tagged adapters using the anti-Flag mAb M2 (Sigma), or anti-GFP mAb (Clontech), which also recognizes the spectral variants of GFP.

### Example 1

### LPS and dsRNA Activate IRF-3 and IRF-7

The promoters of RANTES and IP-10, like that of IFN-β, contain transcription factor binding elements for NF-κB and IRF-3 (Lin et al., Mol Cell Biol 19: 959-966, 1999; Genin et al., J. Immunol. 164: 5352-5361, 2000). The expression of RANTES and IP-10 represent downstream targets of Toll receptors that are entirely independent of MyD88 expression following stimulation by LPS (which is via the TLR4 pathway) or dsRNA (which is via the TLR3 pathway).

A system for testing the ability of TLR4 and TLR3 pathways to induce RANTES and IP10 secretion was developed. Briefly, bone marrow-derived macrophages from wild type or MyD88-deficient mice were stimulated with LPS (0.1-100 ng/ml), Malp-2 (5 nM), or dsRNA (1-100 μg/ml) for 12 hours. The concentration of RANTES was then measured by ELISA.

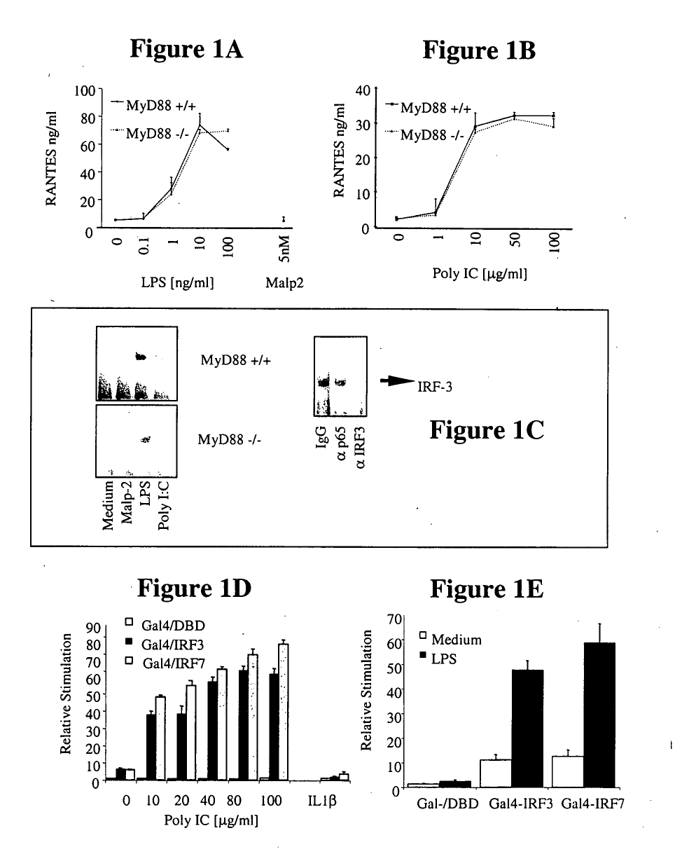

The results of these experiments showed that stimulation of mouse bone marrow-derived macrophages with LPS TLR- or dsRNA TLR-induced RANTES secretion, an effect that was also observed to approximately the same degree in bone marrow macrophages deficient in MyD88 (FIGS. 1A and 1B). This was also observed for IP-10 levels as measured by ELISA. In contrast, TLR2 signaling via lipopeptides requires MyD88 and does not lead to RANTES expression. TLR2 mediated production of TNFα was entirely absent in MyD88-deficient macrophages, which is in agreement with published reports (Kawai et al., Immunity 11: 115-22, 1999; Takeuchi et al., Int. Immunol. 12: 113-17, 2000; Alexopoulou et al., Nature 413: 732-38, 2001). Thus, the mouse bone marrow-derived macrophage system can be used to assay both the MyD88-dependent and the MyD88-independent pathways. This represents a novel method of measuring MyD88-independent signaling.

The effect of LPS and dsRNA on IRF-3 DNA binding activity was examined, which indicates the role of TLR4 and TLR3 pathways, respectively, on IRF-3 binding activity. In these experiments, IRF-3 DNA binding activity was induced in both wild type and MyD88-deficient macrophages following LPS and dsRNA stimulation (FIG. 1C). Activation of IRF-3 is indicated by the presence of IRF-3 in the interferon-stimulated response element (ISRE)-DNA binding complex. This activation was confirmed by depletion (gel shift) analysis using antibodies to IRF-3 (FIG. 1C, lower panel). Stimulation of cells with the TLR2 ligand, Malp-2 did not result in IRF-3 activation. NF-κB was activated in wild type cells by all stimuli and in MyD88-deficient macrophages following LPS or dsRNA stimulation. These experiments illustrate that detecting the activation of IRF-3 is a method of assaying activation of MyD88-independent pathways, i.e., by using MyD88-deficient cells such as macrophages. Test compounds that can affect the activation of IRF-3 in such cells are acting via the MyD88-independent pathway.

The question of whether the transcriptional regulator IRF-7, which is related to IRF-3, is a target of TLR signaling was investigated using an in vivo assay for IRF-7 activation. The assay used a hybrid polypeptide consisting of the yeast Gal4 DNA binding domain (DBD) fused to IRF-7 sequence lacking its own DNA binding domain (Maniatis et al., Cold Spring Harb. Symp. Quant. Biol. 63: 609-20, 1998). Reporter gene expression from the Gal4 upstream activation sequence in this assay requires IRF-7 activation (Wathelet et al., Mol. Cell 1: 507-18, 1998). IRF-3 activation was also measured in this assay using a Gal4-IRF-3 fusion polypeptide. Stimulation of TLR3 or TLR4/MD2-expressing HEK293 cells with dsRNA or LPS but not IL1β activated both IRF-3 and IRF-7 (FIGS. 1D and 1E). These data demonstrate that TLR's can activate IRF-7.

Accordingly, modulation of IFR-7 activation can be achieved by TLR (e.g., TLR4) signaling and IRF-7 can be assayed as part of a scheme to identify compounds that affect TLR signaling. The above method of assaying IRF-7 is novel and is useful, e.g., for identifying compounds that affect IRF-7 activation. Similarly, the corresponding assay using a GAL4-IRF-3 fusion polypeptide can be used to identify compounds that affect IRF-3 activation.

IRF7 plays a critical role in regulating IFN-α1 expression. Accordingly, the ability of IRF7 to activate an IFN-α reporter construct was investigated. It was found that exogenously expressed IRF7 increased the activation of an IFN-α 1 reporter construct when TLR4/MD2 or TLR3-expressing HEK293 cells were stimulated with LPS or dsRNA, while a dominant negative IRF7 mutant inhibited the effect. These observations are strong evidence that TLR3 and TLR4 activate IRF-3 and IRF-7 and as a result, induce IRF-target genes such as RANTES and IFN α/β.

Thus, the experiments described above show that IRF-3, IRF-7, RANTES, and IFNα/β are all potential targets for compounds modulating TLR4 and TLR3 pathways. In particular, IRF-7 is a novel target. The experiments also demonstrate assays that can be used to assay test compounds for their ability to act at or upstream of IRF-3, IRF-7, RANTES, or IFNα/β.

### Example 2

### Identification of a Fourth TIR Domain Containing Adapter Molecule, TRAM

Cloning and Structural Information

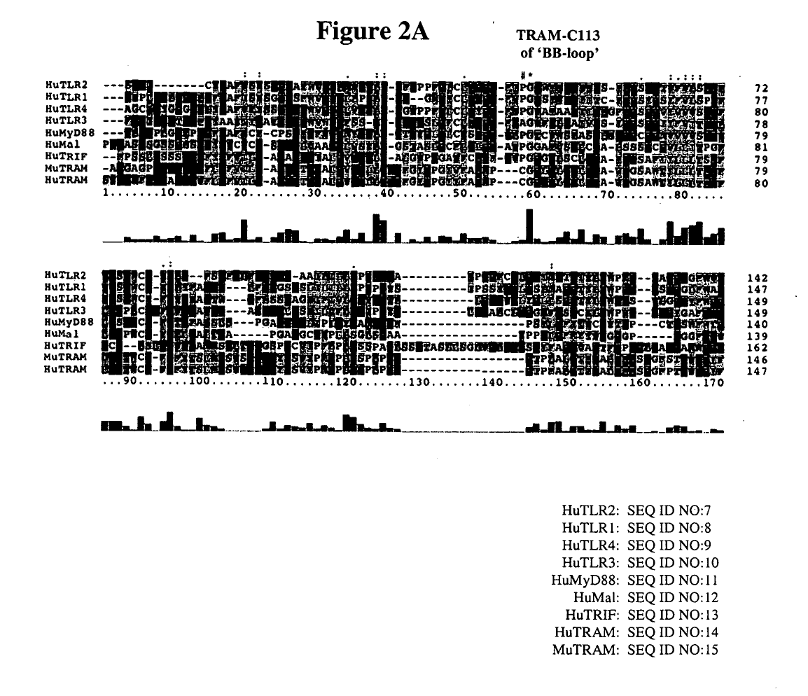

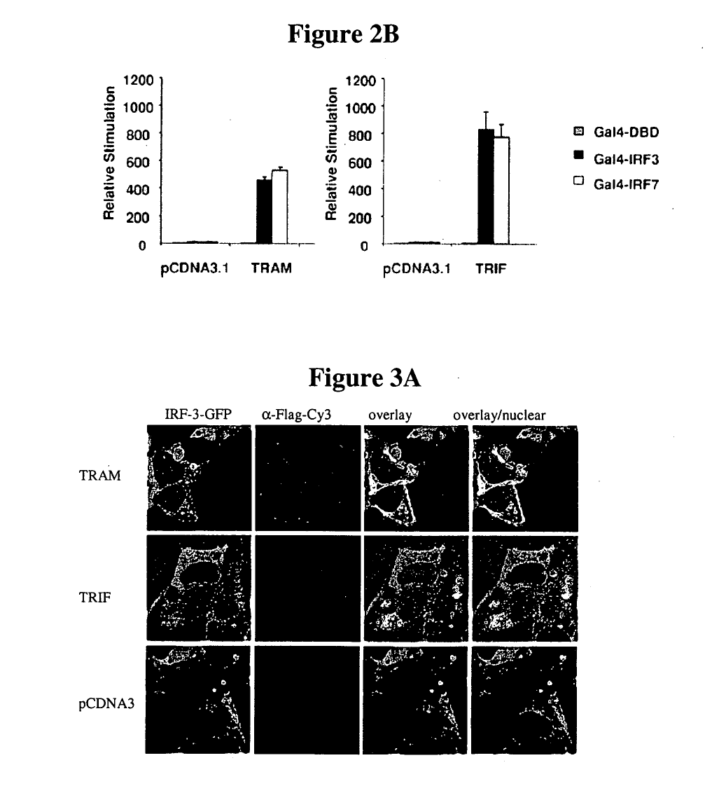

To identify additional components of the TLR pathways, a search of the human genome for previously unidentified molecules containing TIR domains was conducted. This search for TIR domain-containing adapter molecules resulted in the identification of a small polypeptide fragment that shares sequence similarity with other TIR domain-containing polypeptides, most notably with TRIF/TICAM-1. A set of overlapping EST sequences were subsequently identified and used to clone the full-length cDNA of the human and mouse sequences and the predicted polypeptide is termed TRAM (Trif-related adaptor molecule). Human and mouse TRAM share 75% sequence identity (Genbank Accession numbers AY232653 and AY268050, respectively). The TRAM gene was localized to human chromosome 5 (ENSEMBL ID: ENSG00000164226). Human TRAM is a 235 amino acid polypeptide and murine tram is a 232 amino acid polypeptide. Both TRAMs contain a C-terminal TIR domain. FIG. 2a shows a multiple sequence alignment of human and mouse TRAM with other human adapters and TLRs.

The crystal structure of the TIR domain of TLR2 has been resolved. The TIR-domain ‘BB-loop’ of TLR2 is an essential part of its structure, and this portion of the molecule appears to engage downstream elements such as adapter molecules or other TLRs (Xu et al., Nature 408: 111-115, 2000; Dunne and O'Neill, Sci. STKE 171: re3, 2003). Most TIR-domain BB-loop sequences contain a conserved proline residue in the BB-loop. When this residue is mutated to histidine, the mutant polypeptide is typically unable to signal, and may even function as a dominant suppressing mutation (Poltorak et al., Science 282: 2085-88, 1998; Fitzgerald et al., Nature 413: 78-83, 2001; Horng et al., Nat. Immunol. 2: 835-41, 2001). Unlike the other known adapter polypeptides, both human and mouse TRAM contain a cysteine residue at this position (denoted by a # in FIG. 2a). A proline residue resides directly adjacent to this residue in TRAM, at position 116. This difference between the BB-loop regions of TRIF and TRAM also indicates that this site is useful as a specific target site for reagents such as antibodies that can selectively bind to TRIF or TRAM. For example, the BB-loop region can be used to generate a monoclonal antibody that can distinguish between TRIF and TRAM. It also provides a site for targeting compounds that specifically modulate TRAM activity. As such, TRAM polypeptides that include the BB-loop or a portion thereof that contains position 116 are useful as, e.g., peptides for generating TRAM-specific antibodies.

The full sequence of human TRAM appears below. The sequence has a predicted myristoylation site (underlined):

(SEQ ID NO:3)

MGIGKSKINSCPLSLSWGKRHSVDTSPGYHESDSKKSEDLSLCNVAEHSN
TTEGPTGKQEGAQSVEEMFEEEAEEEVFLKFVILHAEDDTDEALRVQNLL
QDDFGIKPGIIFAEMPCGRQHLQNLDDAVNGSAWTILLLTENFLRDTWCN
FQFYTSLMNSVNRQHKYNSVIPMRPLNNPLPRERTPFALQTINALEEESR
GFPTQVERIFQESVYKTQQTIWKETRNMVQRQFIA

The myristoylation signal is GIGKSKINSCPLSLSWG (SEQ ID NO:4). The underlined G residue at amino acid position 2 in the TRAM sequence is the critical position in the myristoylation signal sequence. Mouse TRAM (e.g., Genbank accession no. AY268050) is a 232 aa polypeptide, and also has a predicted myristoylation site (underlined):

(SEQ ID NO:6)

MGVGKSKLDKCPLSWHKKDSVDADQDGHESDSKNSEEACLRGFVEQSSGS
EPPTGEQDQPEAKGAGPEEQDEEEFLKFVILHAEDDTDEALRVQDLLQND
FGIRPGIVFAEMPCGRLHLQNLDDAVNGSAWTILLLTENFLRDTWCNFQF
YTSLMNSVSRQHKYNSVIPMRPLNSPLPRERTPLALQTINALEEESQGFS
TQVERIFRESVFERQQSIWKETRSVSQKQFIA

The predicted myristoylation signal is GVGKSKLDK CPLSWHKK (SEQ ID NO:5). The underlined G residue at amino acid 2 in the sequence is a critical position in the sequence.

The related adapter molecules Mal, MyD88, and TRIF do not have a predicted myristoylation domain.

Additional TRAMs can be identified by one or more of the following features: at least 75% sequence homology with mouse or human TRAM, a myristoylation site at the N terminus, a TIR domain, a cysteine residue in the BB-loop region that in other TIR-domain-containing polypeptides is a proline, and a proline residue adjacent to the cysteine residue.

Effects of TRAM and TRIF on Activation of IRF-3 and IRF-7

Because TRAM and TRIF have similar TIR domains, the effects of TRIF and TRAM on activation of the transcription factors IRF-3 and IRF-7 were investigated. In these experiments, HEK293 cells were transfected as described above and cotransfected with 40 ng of TRAM or TRIF construct. After 24 hours, luciferase reporter gene activity was measured. Overexpression of TRAM activated the IRF-3 response and the IRF-7 response (FIG. 2B). TRIF also activated both transcription factors (FIG. 2B). As a consequence, TRAM and TRIF also induced the IFN-β, RANTES, IP-10 and IFN-α1/α2 promoters, all of which contain interferon-stimulated response elements (ISRE).

These data imply that TRAM and TRIF also activate NF-κB, as some of these promoters (IFN-β, RANTES and IP-10) also require NF-κB for full activity (infra). In addition, these data also show that TRAM and TRIF are positioned upstream of IFN-β, RANTES, IP-10, and IFN-α1/α2 promoters as well as NF-κB and therefore are useful targets for compounds that modulate pathways involving these components.

Effects of TRAM and TRIF on Nuclear Localization

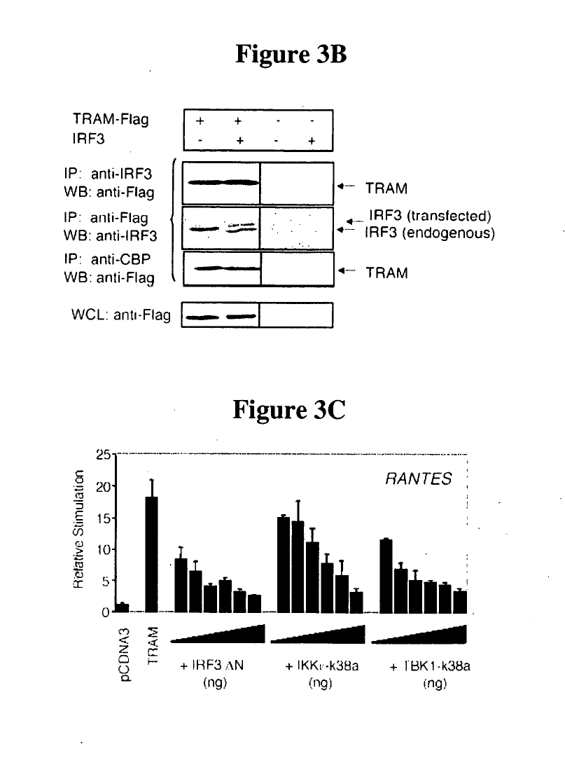

As a further test of TRAM and TRIF-dependent IRF-3 activation, the effects of TRAM and TRIF on the nuclear translocation of IRF-3 were examined. Overexpression of TRAM and TRIF in a stable cell line expressing a green fluorescent polypeptide (GFP) chimera of IRF-3 resulted in the nuclear translocation of this IRF-3-GFP fusion polypeptide (FIG. 3a). TRIF co-immunoprecipitates with IRF-3 (Yamamoto et al., Nature 420: 324-29, 2002). Thus, a compound that interacts with TRAM can, for example, also be tested for its ability to inhibit IRF-3 localization to the nucleus as part of an assay system for identifying compounds that affect specific pathways in the TLR systems.

TRAM interacts with IRF-3 and CBP and Signals via IKKε and TBK1

Since it was found that TRAM can activate IFF-3, further experiments were performed to determine whether TRAM associates with IRF-3. In these experiments, HEK293T cells were transfected with 4 μg of Flag-TRAM with or without a plasmid encoding IRF-3 (untagged) as indicated. Twenty-four hours later, whole cell lysates were immunoprecipitated with anti-IRF-3, anti-Flag, or anti-CBP and the immunoprecipitated complexes immunoblotted for Flag-tagged TRAM and IRF-3. Whole cell lysates (WCL) were also analyzed for Flag-tagged polypeptides.

In the experiments in which HEK293 cells were transfected with Flag-tagged TRAM and immunoprecipitated with antibody to endogenous IRF-3 and the immune complexes examined for the presence of Flag-tagged TRAM, Flag-tagged TRAM was detected in the immunoprecipitated complex (FIG. 3b, top panel). Immunoprecipitation with an anti-Flag antibody confirmed this interaction; endogenous and co-transfected IRF-3 was detected in the immunoprecipitated complexes (FIG. 3b, second panel). TRIF also interacted with endogenous and transfected IRF-3, in agreement with published reports. There were no non-specific associations detected in cells lacking the transfected adapter constructs. In similar experiments using IRF-7, it was found that IRF-7 also interacted with TRAM and TRIF and vice versa.

Activated IRF-3 must associate with the co-activators CBP and p300 to enhance target gene expression (Lin et al., Mol. Cell. Biol. 18: 2986-96, 1998; Wathelet et al., Mol. Cell 1: 507-18, 1998; Weaver et al., Mol. Cell. Biol. 18: 1359-68, 1998; Yoneyama et al., Embo. J. 17: 1087-95, 1998). When endogenous CBP was immunoprecipitated from cell lysates expressing transfected Flag-tagged TRAM, TRAM could be detected in these immunoprecipitated complexes (FIG. 3b, third panel). This was also the case for transfected TRIF. These data demonstrate that TRAM can be in a complex that contains at least IRF-3, CBP, and p300.

Thus, compounds that disrupt the interaction between IRF-3 and TRAM or TRIF, or IRF-7 and TRAM or TRIF are useful for decreasing activation of the immune system in pathways that utilize IRF-3 or IRF-7, e.g., pathways that are mediated by TLR4 or TLR3. Similarly, compounds that disrupt the interaction between TRAM or TRIF and CBP or p300 are also useful for decreasing activation of TLR signaling pathways.

Effect of IKKε and TANK Binding Kinase on TRAM Signaling

The non-canonical IκB kinases, IκB kinase-ε (IKKε) (Shimada et al, Int. Immunol. 11: 1357-1362, 1999; Peters et al., Mol. Cell 5: 513-22, 2000) and TANK binding kinase 1 (TBK1) (Pomerantz, J. L., and D. Baltimore, Embo. J. 18: 6694-704, 1999; Bonnard et al., Embo. J. 19: 4976-85, 2000) are key regulators of the IRF-3 activation pathway resulting from viral exposure and activation of TLR3 or TRIF signaling cascades (Fitzgerald et al., Nat. Immunol. 4: 491-496, 2003; Sharma, tenOever et al. 2003). IKKε has also been implicated in LPS signaling (Kravchenko et al., J. Biol. Chem. 278: 26612-619, 2003). The effect of dominant negative mutants of IKKε and TBK1 on TRAM signaling was examined. A RANTES reporter gene construct was used to examine the relationship between the IκB kinases IKKε and TBK1 on TRAM.

In these experiments, HEK293 cells were transfected with the RANTES luciferase reporter gene and TRAM (20 ng), which activates downstream molecules by overexpression, and cotransfected with increasing concentrations of the kinase-inactive mutants of IKKε (IKKε-k38a), TBK1 (TBK1-k38a), or IRF-3-ΔN constructs at 10, 20, 30, 40, 60, or 80 ng. Luciferase reporter gene activity was measured 24 hours after transfection. Both mutants inhibited TRAM-induced RANTES reporter activation in a dose-dependent manner. This shows that these two kinases may also function downstream of TRAM. The observations reported herein show that TRAM and TRIF are important components of the IRF-3 signaling pathway, and suggest that these adapter polypeptides form a multi-polypeptide complex with IRF-3/7, CBP and the IRF-3/7 kinases (IKKε and TBK1) during signal transduction.

Compounds that disrupt the association of TRAM or TRIF with these multipolypeptide complexes are useful for, e.g., inhibiting immune system pathways. In addition, compounds that modulate the association of TRAM or TRIF with a multipolypeptide complex will also modulate the activation of other components of the pathway, e.g., IRF-3, IRF-7, CBP, and IRF-3/7 kinases.

### Example 3

### TRAM Activates the IRF Pathway in the TLR4, but not the TLR3, Signaling Pathway, and TRAM Mediates the TLR4 Pathway to IRF-3 and IRF-7

To examine the relationship between the structure and functional activity of TRAM, a series of TRAM mutants were generated and their ability to activate the RANTES reporter gene was evaluated.

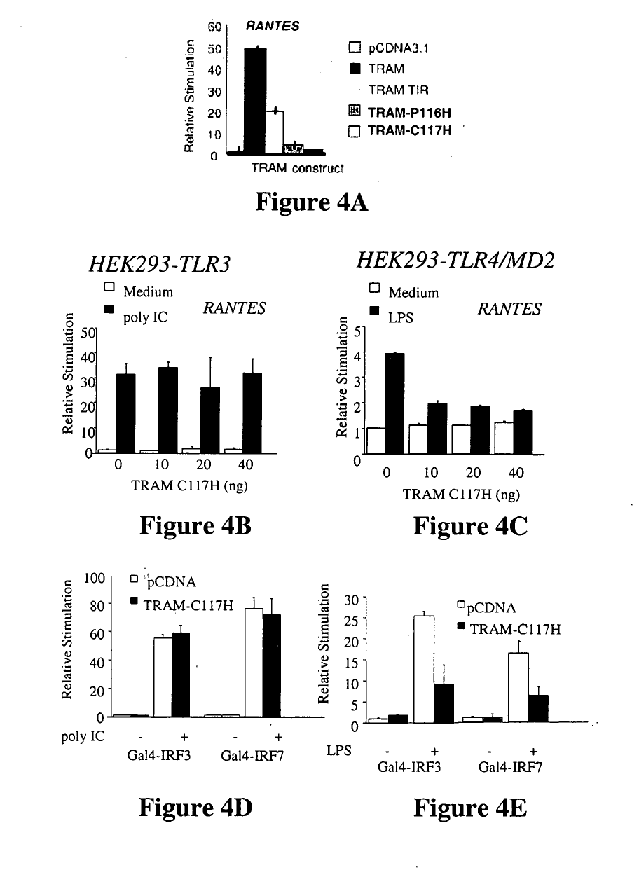

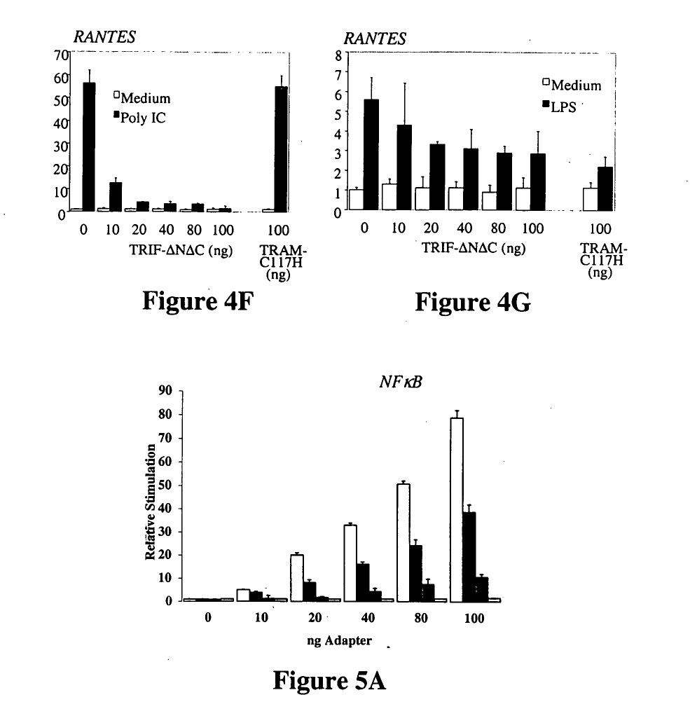

In some experiments, HEK293 cells were transfected with 40 ng of a RANTES reporter construct and cotransfected with TRAM, TRAM-TIR (the TIR domain of TRAM alone), TRAM-C117H or TRAM-P116H. The TRAM-TIR construct induced the RANTES reporter, although this response was considerably less than that observed with the full length TRAM cDNA (FIG. 4a).

As discussed above, TRAM contains a cysteine residue (C117) in the BB-loop with an adjacent proline residue (P116). When TRAM constructs containing a mutation of the proline residue to histidine (TRAM-P116H) were co-transfected into cells containing the RANTES reporter, the RANTES inducing activity of TRAM was significantly impaired. Mutation of the cysteine residue at position 117 (TRAM-C117H) completely abrogated all activity (FIG. 4a). Thus, either TRAM-C117H or TRAM-P116H can function as a dominant interfering mutant of TRAM activity. The effect of these TRAM constructs was similar when an IP-10 promoter-based reporter construct was assessed.

Next, the effect of the TRAM mutants on signaling upstream of RANTES and IP-10 was investigated. This was done by examining the effects of the TRAM mutants on TLR-mediated signaling that culminates in RANTES promoter activation or the activation of the transcription factors IRF-3 and IRF-7. The experiments focused on TLR3 and TLR4 because of their unique abilities to activate both NF-κB and IRF-3. Briefly, TLR4/MD2- and TLR3-expressing HEK293 cell lines were transfected with a luciferase reporter gene containing the Gal4 upstream activation sequence and cotransfected with Gal4-DBD, Gal4-IRF-3, or Gal4-IRF-7 or the RANTES luciferase reporter gene as well as TRAM-C117H or TRIF-ΔNΔC. On the following day, cells were stimulated with LPS (10 ng/ml), dsRNA (50 μg/ml poly I:C), or left untreated for about 8 hours and luciferase reporter gene activity measured.

Neither the TRAM-TIR domain nor the TRAM-P116H mutants had any dominant negative inhibitory activity on either TLR-dependent IRF-3 pathway tested. Transfection of HEK/TLR3 cells with TRAM-C117H had no inhibitory effect on dsRNA-induced RANTES response (FIG. 4B). In contrast, LPS-induced activation of the RANTES reporter via TLR4 was impaired by the TRAM-C117H mutant (FIG. 4C). The LPS-dependent induction of the RANTES reporter gene was considerably less potent than that observed following TLR3 stimulation. The TRAM-C117H mutant also inhibited the TLR4- but not the TLR3-dependent activation of IRF-3 and IRF-7 (FIGS. 4D-E). The TRAM-C117H mutant also inhibited the TLR4- but not the TLR3-dependent activation of an IP-10 reporter construct.

The role of TRIF in the TLR3- and TLR4-dependent pathways in parallel was examined by expressing a dominant negative mutant of TRIF lacking both the N-terminal and C-terminal regions surrounding the TIR domain (TRIFΔNΔC (Yamamoto et al., Nature 420: 324-329, 2002)). As expected, this mutant completely suppressed the TLR3-dependent response (FIG. 4F). The TRIFΔNΔC mutant also inhibited the TLR4-response, although the effect was less potent than that observed in the TLR3 pathway under identical experimental conditions (FIG. 4G).

Taken together, these observations show that TRIF regulates the TLR3 and TLR4 pathways to IRF-target genes, while TRAM appears to be TLR4 specific. Therefore, compounds that modulate TRAM are useful for specifically targeting modulation of TLR4-specific signaling pathways while compounds that modulate TRIF are useful for modulating both TLR4 and TLR3 signaling pathways.

### Example 4

### TRAM Activates NF-κB and is Specific to the TLR4 Pathway

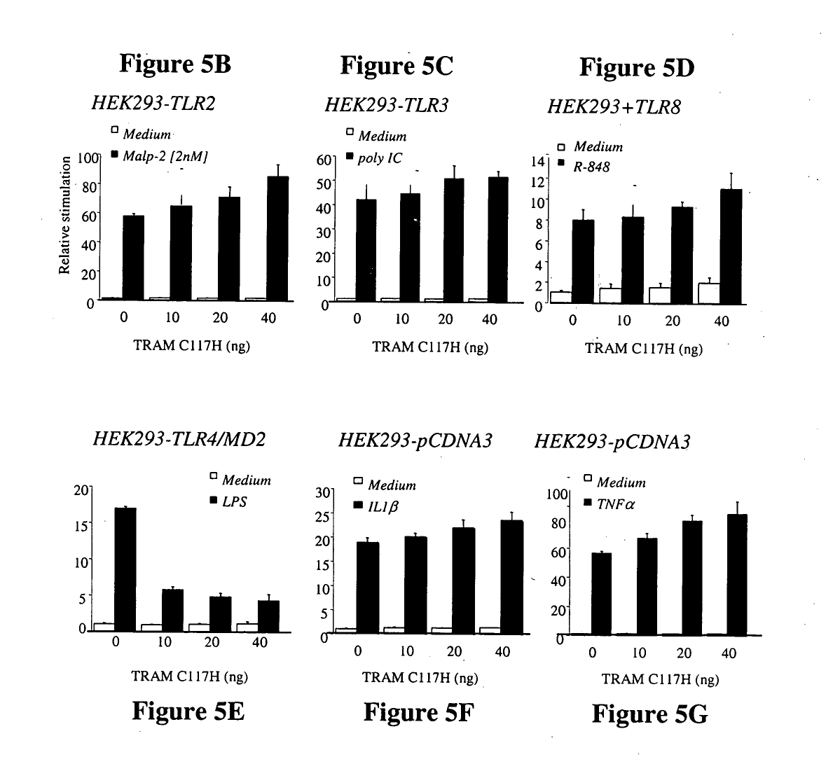

The role of TRAM in the NF-κB activation pathway was examined further. First, experiments were conducted in which HEK293 cells were transfected with 40 ng of an NFκB reporter construct and cotransfected with TRAM, TRAM-TIR, TRAM-C117H, and TRAM-P116H. In these experiments transfection of HEK293 cells with TRAM resulted in a potent NF-κB activation response (FIG. 5A). The isolated TIR domain of TRAM also induced a robust NF-κB response, though this was considerably less than that observed with the full-length gene (FIG. 5A). Neither the TRAM-P116H nor the TRAM-C117H mutants induced NF-κB activation. Thus, like all of the other known TLR adapters, TRAM is also an NF-κB activator.

Effect of a TRAM Mutant on TLR-Dependent Signaling to NFκB

The TRAM-C117H negative interfering mutant was next tested for its ability to inhibit TLR-dependent signaling to NF-κB. TLR2-, TLR3-, TLR4/MD2-, TLR7-, and TLR8-expressing HEK293 cells were transfected individually with 40 ng of an NF-κB reporter gene and co-transfected with increasing concentrations of TRAM-C117H. One day after transfection, TLR-expressing cells were stimulated with Malp-2 (2 nM), dsRNA (100 μg/ml poly I:C), LPS (10 ng/ml), R-848 (10 μM), IL1β (10 ng/ml), TNFα (10 ng/ml), or left untreated for 8 hours and luciferase reporter gene activity was measured.

NF-κB activation induced by the TLR2 agonist Malp-2, the TLR3 agonist dsRNA, the TLR7 and TLR8 agonists, R-848, IL-1β or TNFα were all unaffected when cells were co transfected with the suppressing TRAM-C117H mutant. In contrast to these negative results, the TRAM-C117H mutant inhibited LPS-induced NF-κB activity in HEK/TLR4/MD2 cells. The TRAM-P116H had no inhibitory activity on any TLR-pathway to NF-κB, including the TLR4 pathway, confirming the importance of the Cl 17 residue for this response (see FIGS. 5B-G).

These observations demonstrate that TRAM regulates NF-κB as well as IRF-3/7 in the LPS/TLR4 signaling pathway. Accordingly, compounds that modulate TRAM activity also affect NF-κB activation, which can be used as a supplemental assay for determining the efficacy of compounds that bind to TRAM, or affect TRAM expression or activity (e.g., by binding to TRAM).

### Example 5

### TRIF and TRAM Cooperate in the IRF-3 Activation Pathway—TRAM Signaling Requires the Expression of TRIF

The effect of the TRIFΔNΔC mutant on TRAM-induced RANTES promoter activation was examined to define the relationship between TRIF, TRAM, and the TLR4 pathway.

In these experiments, HEK293 cells were transfected with the RANTES luciferase reporter gene and TRAM or TRIF (40 ng) and co-transfected with TRIF-ΔNΔC or TRAM-C117H. Luciferase reporter gene activity was measured 24 hours later.

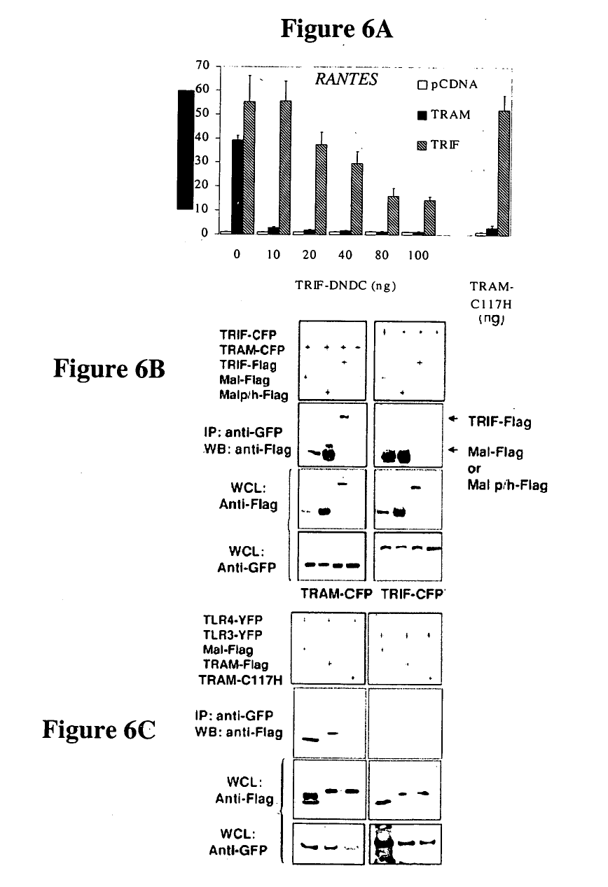

The TRIFΔNΔC construct inhibited the TRIF-induced RANTES reporter gene response (FIG. 6A, hatched bars). The TRIFΔNΔC mutant completely abrogated the TRAM-induced RANTES reporter gene response (FIG. 6A). The TRAM-C117H mutant also abrogated the induction of the RANTES reporter gene in response to TRAM overexpression (FIG. 6A, far right), but had no effect on the response to TRIF overexpression (hatched bars). The observation that a TRIF dominant-negative construct blocked TRAM activity but not vice versa suggests that TRAM signaling requires TRIF. Thus, compounds that interfere with TRIF signaling also interfere with TRAM signaling, however, compounds that interfere with TRAM signaling do not interfere with TRIF signaling. This further demonstrates that TRAM is a useful target for identifying compounds that selectively modulate certain TLR signaling pathways (e.g., TLR4 and TLR3).

Co-immunoprecipitation experiments were performed using cells that heterologously expressed either of TRAM or TRIF, as well as the related adapter molecule Mal/TIRAP. In these experiments, 293T cells were transfected with 4 μg of TRAM-CFP or TRIF-CFP and co-transfected with Flag-Mal, Flag-Mal-P125H, or Flag-TRIF. Whole cell lysates were harvested 48 hours later, and immunoprecipitated with anti-GFP antibody (which also immunoprecipitates cyan fluorescent polypeptide; CFP or yellow fluorescent polypeptide; YFP). Immunoprecipitated complexes were resolved by SDS-PAGE and immunoblotted for Flag-tagged adapters. Whole cell lysates (WCL) were also analyzed for CFP- and Flag-tagged polypeptides by immunoblotting.

Western blotting of lysates demonstrated expression of stable TLRs and transfected adapter polypeptides. These immunoprecipitation studies revealed that TRAM interacted with both TRIF and Mal/TIRAP (FIG. 6B, left panel). TRIF also interacted with Mal (6B, right panel). Finally, both TRIF and TRAM interacted with the Mal-P125H (dominant negative) mutant. The stronger interaction of TRIF and TRAM observed with the Mal-P125H mutant does not reflect a higher avidity for this interaction, but rather was a consequence of the higher expression level of the MAL-P125H mutant in whole cell lysates, compared to the expression level of Mal or TRIF (FIG. 6B). These data may explain a previously unexplained finding, i.e., that the Mal/TIRAP dominant negative mutant powerfully inhibited LPS-induced signaling to NF-κB (Fitzgerald et al., Nature 413: 78-83, 2001; Horng et al., Nat. Immunol. 2: 835-41, 2001) and IFN-β expression (Shinobu et al., FEBS Lett. 517: 251-56, 2002; Toshchakov et al., Nat. Immunol. 3: 392-398, 2002), while the Mal/TIRAP knockout mouse both retained the ability to induce NF-κB (Horng et al., 2002, supra; Yamamoto et al., Nature 420: 324-329, 2002) and IFN-β expression (Yamamoto et al., 2002, supra). The more profound effect of the dominant negative construct is likely to be due to its ability to limit the function of other adapter molecules involved in LPS signaling such as TRAM and TRIF. Furthermore, these data suggest that TRIF and TRAM interact with Mal at a site distinct from the TLR4 interaction site of Mal (Horng et al., Nat. Immunol. 2: 835-841, 2001). Accordingly, compounds that modulate the interaction between TRAM or TRIF and Mal are useful for modulating TLR signaling pathways. Such compounds generally do not interfere with the TLR4 interaction with Mal.

Interaction of TRAM with TLR4

Co-immunoprecipitation studies were performed to determine if TRAM interacts with TLR4. Stable TLR4^{YFP}- or TLR3^{YFP}-expressing HELA cells were transiently transfected with 4 μg of plasmid encoding Flag-Mal, Flag-TRAM or Flag TRAM-C117H. Forty-eight hours after transfection, whole cell lysates were immunoprecipitated with anti-GFP antibody and immunoprecipitated complexes immunoblotted for Flag-tagged adapters and co-immunoprecipitation experiments performed. Western blotting of the cell lysates demonstrated expression of stable TLRs and transfected adapter polypeptides.

The results of these experiments indicated that TRAM interacts with TLR4, but not with TLR3 (FIG. 6C), one more indication of the specificity of TRAM for the TLR4 pathway. The dominant negative mutant TRAM-C117H, failed to immunoprecipitate with TLR4, suggesting that the C117 residue is critical for this interaction. Mal also interacted with TLR4 and not TLR3, providing additional evidence that Mal has a role in the TLR4 but not the TLR3 signaling pathway.

These data confirm the specificity of TRAM for acting within the TLR4 signaling pathway. They also demonstrate additional targets for compounds that can modulate signaling pathways involving TRAM. For example, since TRAM interacts with TRIF, compounds that disrupt this interaction will disrupt signaling pathways involving TRAM, i.e., the TLR4 pathway. The data also show that TRAM-C117 is a useful target for specifically interfering with the interaction between TRAM and TRIF. Mal is also a TRAM-interacting polypeptide that falls within this category.

### Example 7

### siRNA Silencing Confirms TRIF and TRAM are Essential for TLR4 Signaling (RANTES Activation by LPS)

The data obtained by testing dominant negative constructs and assessing polypeptide: polypeptide interactions show that TRIF and TRAM both function in the TLR4 signal transduction pathway. Dominant negative constructs, when highly expressed, have the potential to bind (e.g., as seen in FIG. 6b) and interfere with polypeptides that might otherwise not be related to a specific signal transduction pathway. Therefore, siRNA silencing experiments were performed as an additional methodology to delineate the relationship between TRIF and TRAM in the TLR4 and TLR3 signaling pathways. In these experiments, 293T cells plated in 24-well plates were transiently transfected with 1 μg of plasmids encoding fluorescent chimeric constructs of TRAM-CFP, TRIF-CFP, or Mal-CFP and cotransfected with 50 nM siRNA-TRAM or, as a control, Lamin A/C. Twenty-four hours later, CFP fluorescence was measured by flow cytometry using a 405 nm laser for excitation of CFP. The siRNA duplexes were used to assess the effect of silencing a fluorescent chimeric construct of TRAM. This methodology has been used extensively to assess siRNA efficiency and provides a quantitative assessment of silencing efficiency (Brummelkamp et al., Science 296: 550-53).

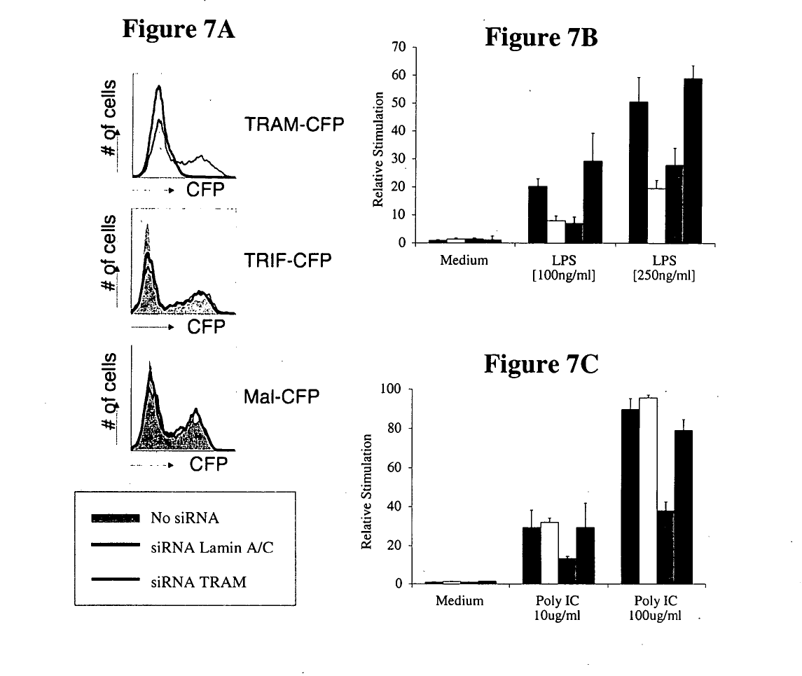

These experiments demonstrated that siRNA duplexes targeting the TRAM coding region completely ablated the expression of the TRAM-CFP chimeric fusion polypeptide while lamin A/C siRNA duplexes were without effect (FIG. 7A). The effect of the TRAM siRNA duplexes on TRIF and Mal expression were investigated to insure the specificity of the TRAM siRNA duplexes. This is particularly important as TRIF and TRAM are most closely related in sequence. TRAM siRNA duplexes had no effect on chimeric constructs of TRIF or Mal expressed as CFP fusion polypeptides (FIG. 7A). These data also demonstrate that siRNA can be used to specifically modulate expression of TRAM.

Having determined that the siRNA duplexes chosen for TRAM effectively and specifically suppressed TRAM expression, the effect of these siRNA duplexes on the LPS and dsRNA signaling pathways was examined. Native macrophages and macrophage cells lines are extraordinarily difficult to transfect with siRNA. In contrast, U373/CD14 cells resemble CNS macrophages, are easily transfectable and are highly inducible by treatment with LPS. Therefore, the effect of TRAM siRNA duplexes on the LPS-response in U373-CD14 cells was tested. For comparison, HEK/TLR3 cells were used to test the effects of TRAM and TRIF in pI:C stimulated RANTES expression. The response of each of these cell lines to these TLR ligands is comparable. In these experiments, U373/CD14 or TLR3-expressing HEK293 cells were transfected with a RANTES reporter gene and co-transfected with siRNA duplexes for 36 hours. Cells were then stimulated for 8 hours with LPS or dsRNA, and luciferase reporter gene activity was measured.

TRAM siRNA duplexes inhibited the LPS-dependent induction of the RANTES reporter gene in U373/CD14 cells, while siRNA targeting of Lamin A/C had no effect (FIG. 7b, top panel). The effect of reported TRIF siRNA duplexes was also examined. The TRIF siRNA duplexes target the 3′ untranslated region of TRIF. These TRIF siRNA duplexes have been shown to completely silence endogenous TRIF mRNA expression (Oshiumi et al., Nat. Immunol. 4(2): 161-7, 2003). TRIF siRNA duplexes also inhibited the LPS response to RANTES induction (FIG. 7b, top panel). In striking contrast to LPS, when the TLR3-mediated response to dsRNA was analyzed, the TRAM siRNA duplexes had no inhibitory effect on the dsRNA response, while TRIF siRNA duplexes inhibited dsRNA-dependent RANTES induction, in agreement with published reports (Oshiumi et al., 2003, supra). As with RANTES, siRNA silencing of TRAM prevented LPS but not poly IC induction of the IP-10 promoter.

To further confirm the specificity of the action of TRAM in the TLR4 signalling pathway, thioglycollate-elicited peritoneal macrophages were isolated from mice lacking the TRAM gene (Yamamoto et al., Nature Immunol. 4(11): 1144-50, 2003, transfected with a RANTES reporter gene, and cultured with LPS, heat-killed E. Coli (gram negative bacteria), heat-killed group B streptococcus (gram positive), R848 (a TLR7 agonist, GL Synthesis, Worcester, Mass.), Sendai Virus (a non-TLR activating pathogen, Charles River Laboratories, Wilmington, Mass.), or CpG DNA (a TLR9 agonist, MWG Synthesis, High Point, N.C.). FIG. 9 shows the results of these experiments. Signalling was measured by induction of the RANTES reporter gene. There was a significant reduction in signalling in the TRAM knockout cells, as compared to signalling in wild type cells isolated from C57/BL6 mice, when TLR4 agonists were used (LPS, gram negative bacteria) but not when non-TLR4 agonists were used (e.g., agonists of TLR2, -7, or 9 or non-TLR pathogens). This provides additional evidence of the specificity of TRAM activity in the TLR4 pathway.

These observations confirm the studies with TRIF and TRAM dominant negative mutants and demonstrate that both adapter molecules are required for full LPS/TLR4 signaling to IRF target genes, as well as the specificity of TRAM for TLR4 signaling. They also demonstrate that siRNAs or other compounds that specifically target TRAM (e.g., by modulating TRAM expression) are useful for modulating TLR4 signaling, and siRNAs or other compounds that specifically target TRIF (e.g., by modulating TRIF expression) are useful for modulating TLR4 signaling.

### Example 8

### TRAM Contains a Membrane Targeting Myristoylation Sequence

Both human and mouse TRAM were predicted to contain an N-terminal myristoylation site. To test the subcellular localization of TRAM, a plasmid encoding a fluorescent fusion polypeptide of TRAM-fused in-frame with cyan fluorescent polypeptide (CFP) was generated (TRAM-CFP) and transfected into HEK293 cells. Microscopy was used to determine the subcellular location of the TRAM-CFP.

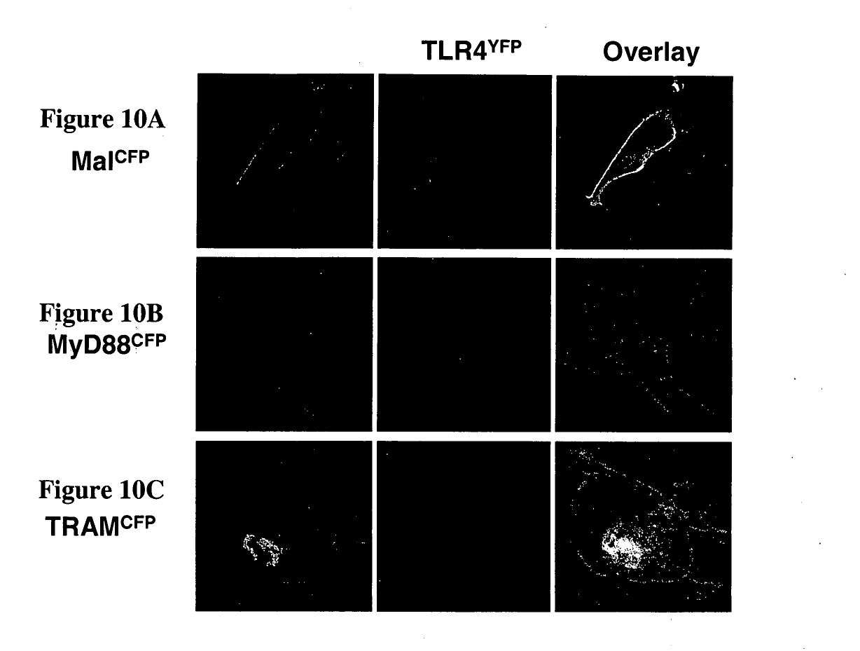

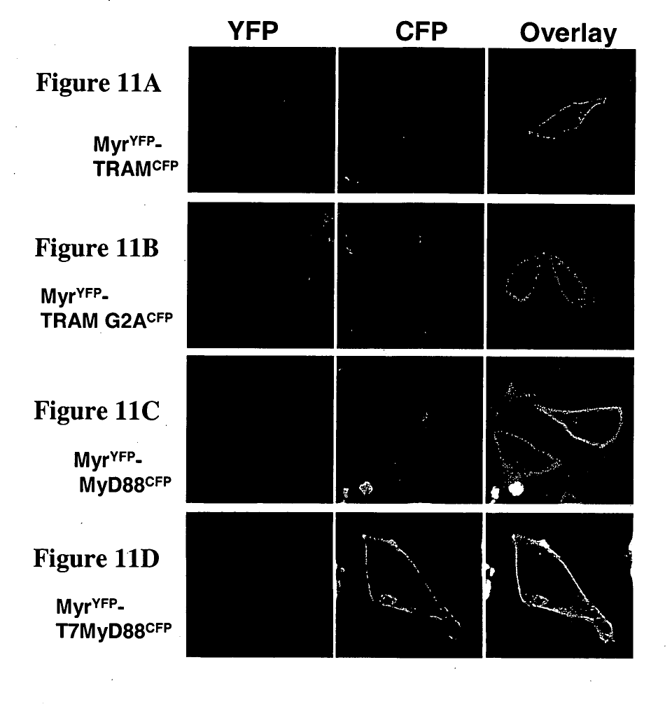

TRAM-CFP was observed to be localized to the plasma membrane in the transfected HEK293 cells, colocalizing with a yellow fluorescent polypeptide (YFP)-tagged glycosyl-phosphatidylinositol (GPI) membrane anchor (not shown) and a membrane marker fusion polypeptide of the myristoylation sequence from Src kinase fused to YFP (Myr-YFP) (FIG. 10A and 11A), which is known to localize to the plasma membrane. The LPS receptor TLR4 is located primarily on the surface of cells in addition to the Golgi apparatus (FIGS. 10A-C, middle panels). The localization pattern of TRAM is punctate and may be RAFT localized (FIG. 10C). As a control, the subcellular localization of MyD88 and Mal, related adaptor polypeptides believed to act in the TLR4 pathway, was tested using MyD88-CFP (FIG. 10B) and Mal-CFP (FIG. 10A) fusion polypeptides. The Mal-CFP construct localized to the plasma membrane, colocalizing with TLR4 (FIG. 10A). In addition, GST-pull down experiments have demonstrated that Mal and TRAM bind directly to each other. In contrast, MyD88-CFP (which lacks a predicted myristoylation sequence) is localized to the cytoplasm of HEK293 transfected cells (FIG. 10B, 11C).

To examine the role of the myristoylation site of TRAM, stable cell lines were generated that expressed CFP fusion polypeptides with TRAM containing a mutation of the G residue that is critical for myristoylation signaling to an alanine (TRAM-CFP-G2A). Introduction of this mutation into the myristoylation site resulted in a form of TRAM that no longer localizes to the surface of cells but localizes to the cytoplasm. Thus, the myristoylation site of TRAM is critical for membrane localization. Introduction of the myristoylation sequence of TRAM (GIGKSKINSCPLSLSWG (SEQ ID NO:4)) into the N-terminus of MyD88 (Myr-MyD88) creates a membrane-localized form of MyD88. As noted above, the WT form of MyD88 fused to CFP was found primarily in the cytoplasm (FIGS. 10C and 11), but the Myr-MyD88 form colocalizes with a membrane marker fusion polypeptide of the myristoylation sequence from Src kinase fused to YFP (10D). This indicates that the myristoylation sequence from TRAM by itself is sufficient to direct MyD88 to the plasma membrane.

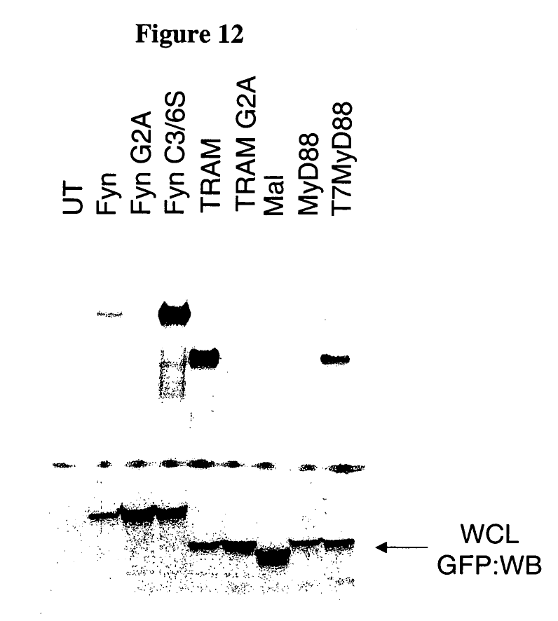

To evaluate the myristoylation state of the various constructs, incorporation of ³H-Myristic acid was measured using standard methods. The results, shown in FIG. 12, demonstrate that TRAM and the Myr-MyD88 mutant are myristoylated in vivo. As controls, fyn polypeptide, which is known to be myristoylated, myristoylated (Fyn C3/6S) and non-myristoylated (Fyn G2A) fyn mutants, and wild type Mal (which is not myristoylated) were used.

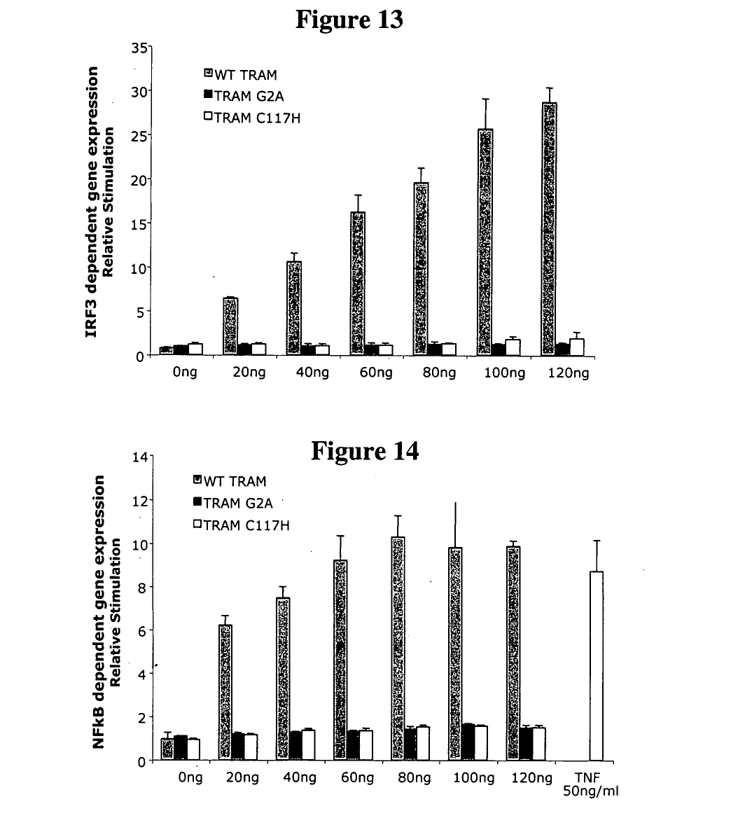

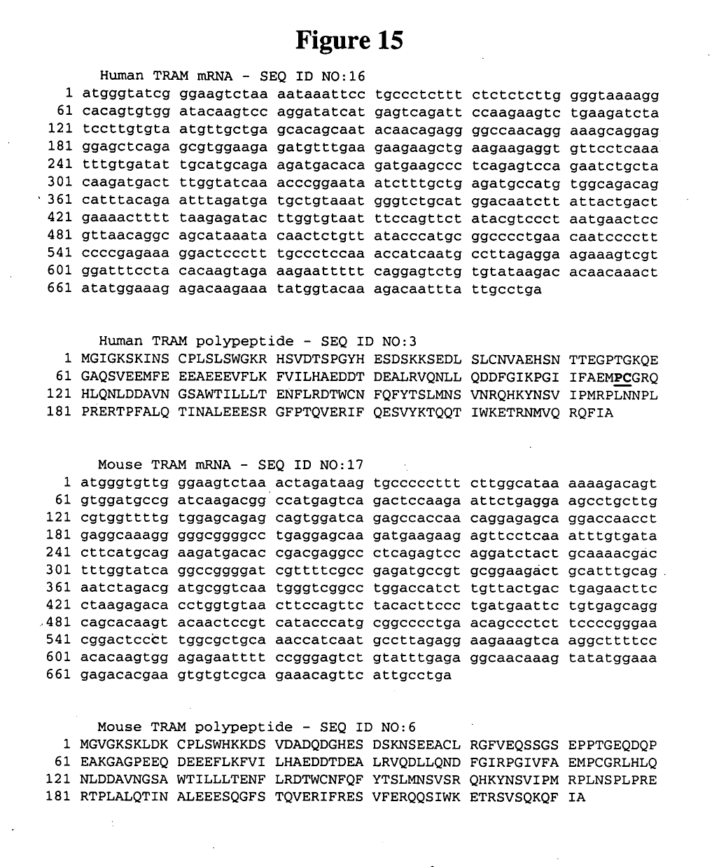

To further investigate the importance of myristoylation to TRAM function, NFkB and IRF-3 dependent gene expression was evaluated in cells expressing the wild type TRAM, the TRAM G2A myristoylation mutant, and the TRAM C117H dominant negative mutant. As is shown in FIGS. 13 and 14, both the G2A and C117H mutants almost completely abolished NFkB and IRF-3 dependent gene expression, indicating that myristoylation is essential for the proper functioning of TRAM.

Compounds that interfere with myristoylation therefore interfere with appropriate localization of TRAM and are useful for inhibiting TRAM signaling. The myristoylation site is therefore a target site for such compounds. Assays for compounds that interfere with localization can performed using methods known in the art and as described herein. For example, by testing the ability of a test compound to disrupt localization of a TRAM-fluorescent fusion polypeptide that is stably expressed in a cell line. Localization of TRAM can be detected using, e.g., microscopy or cell fractionation techniques.

Without committing to any particular model, a likely model of TRAM signaling is illustrated in FIG. 8. In response to LPS stimulation of the cell, TLR4 molecules translocate to the raft, where at least two TLR4 molecules are brought into juxtaposition permitting homodimerization of TRAM, which is already membrane localized by virtue of the myristoylation site. This forms a “platform” to which other adapters, e.g., MyD88 and TRIF are recruited.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated Toll-IL-1-resistance domain-containing adapter-inducing IFN-β-related adapter molecule (TRAM) polypeptide comprising the amino acid sequence of SEQ ID NO:3 or 6, or an active fragment thereof.

2. The polypeptide of claim 1, wherein the active fragment can bind to one or more of Toll-IL-1-resistance domain-containing adaptor-inducing IFN-β (TRIF), Toll-Like Receptor 4 (TLR4), CREB-Binding Polypeptide (CBP), or MyD88 adaptor-like (Mal).

3. The polypeptide of claim 1, wherein the active fragment can form a complex with Mal and Myeloid Differentiation Primary Response Gene 88 (MyD88).

4. The polypeptide of claim 1, wherein the active fragment can induce nuclear factor kappa B (NFkB) or interferon regulatory factor 3 (IRF-3) dependent gene expression in a cell, in response to stimulation of a TLR4 receptor expressed in the cell.

5. The polypeptide of claim 1, wherein the active fragment can inhibit an activity of the full length TRAM polypeptide.

6. An isolated nucleic acid encoding the TRAM polypeptide of claim 1.

7. The isolated nucleic acid of claim 6, comprising the sequence of SEQ ID NO:16 or 18.

8. An oligonucleotide comprising at least about 15 consecutive nucleotides of SEQ ID NO:16 or 18.

9. A method of identifying a candidate compound that modulates an interaction between a Toll-IL-1-resistance domain-containing adaptor-inducing IFN-β-related adaptor molecule (TRAM) and a TRAM-effector, the method comprising:
(a) providing a sample comprising a TRAM polypeptide and a TRAM-effector;
(b) contacting the sample with a test compound; and
(c) determining the level of interaction between the TRAM polypeptide and TRAM-effector in the presence of the test compound as compared to the level of interaction in a control sample;
wherein a difference in the level of interaction indicates that the test compound is a candidate compound for modulating the interaction between TRAM and a TRAM-effector.

10. The method of claim 9, wherein the test compound increases the level of the interaction.

11. The method of claim 9, wherein the test compound decreases the level of the interaction.

12. The method of claim 9, wherein the test compound is an antibody.

13. The method of claim 12, wherein the antibody specifically binds to a site that includes at least one of cysteine 117 or proline 116 of of SEQ ID NO:3, or cysteine 113 or proline 112 of SEQ ID NO:6.

14. The method of claim 9, wherein the TRAM-effector is TRIF, Mal, TLR4, MyD88, CBP, or p300.

15. The method of claim 9, wherein the TRAM and the TRAM-effector are in a cell.

16. A method of identifying a candidate compound that can modulate Toll-IL-1-resistance domain-containing adaptor-inducing IFN-β-related adaptor molecule (TRAM) signaling, the method comprising:
(a) providing a cell that expresses a TRAM polypeptide;
(b) contacting the cell with a test compound;
(c) determining TRAM polypeptide localization in the cell,
wherein a difference in the localization of the TRAM polypeptide in the presence of the test compound compared to a control indicates that the test compound is a candidate compound for modulating TRAM signaling.

17. The method of claim 16, wherein the TRAM polypeptide is a fluorescent TRAM fusion polypeptide.

18. The method of claim 16, wherein the test compound is an inhibitor of myristoylation.

19. A method of modulating the ability of a cell to signal in response to a Toll-Like receptor 4 (TLR4) agonist, the method comprising
(a) providing a cell that can undergo TLR4 signaling; and
(b) contacting the cell with a compound in an amount sufficient to modulate Toll-IL-1-resistance domain-containing adaptor-inducing IFN-β related adaptor molecule (TRAM) expression or activity,
thereby modulating the ability of the cell in response to a TLR4 agonist.

20. The method of claim 19, wherein the compound is an siRNA.

21. The method of claim 19, wherein the compound is an antibody.

22. The method of claim 19, wherein the compound modulates myristoylation of TRAM.

23. The method of claim 19, wherein the compound increases TLR4 signaling.

24. The method of claim 19, wherein the compound decreases TLR4 signaling.

25. The method of claim 19, wherein TLR4 signaling is detected by assaying IFN-β activation, RANTES secretion, or induction of IP10, IP10, IRF1, or IFIT1 (interferon-induced polypeptide with tetratricopeptide repeats 1).

26. A method of detecting Toll-Like Receptor (TLR) signaling, the method comprising
(a) providing a cell that expresses a TLR;
(b) contacting the cell with an inducer of TLR signaling; and
(c) detecting a level of secretion of RANTES, activation of IFN-β, or expression of IP10,
wherein the level of secretion of RANTES, activation of IFN-β, or expression of IP10 indicates the presence of TLR signalling in the cell.

27. The method of claim 26, further comprising contacting the cell with a test compound, and determining the effect of the test compound on TLR signaling in the cell.

28. The method of claim 26, wherein the TLR is TLR3 or TLR4.

29. The method of claim 26, wherein the cell is a bone marrow-derived macrophage.

30. A method of ameliorating an inflammatory response in a cell that is susceptible to or undergoing an inflammatory response, the method comprising contacting the cell with a compound that decreases Toll-IL-1-resistance domain-containing adaptor-inducing IFN-β-related adaptor molecule (TRAM) expression or activity in an amount sufficient to decrease an inflammatory response.

31. The method of claim 29, wherein the compound is selected from the group consisting of a TRAM anti sense oligonucleotide, TRAM siRNA, TRAM morpholino oligonucleotide, anti-TRAM antibody, and a TRAM dominant negative polypeptide.

32. An antibody or antigen-binding portion thereof that specifically binds to a Toll-IL-1-resistance (TIR) domain-containing adaptor-inducing IFN-β (TRIF)-related adaptor molecule (TRAM) polypeptide.

33. The antibody of claim 35, wherein the antibody specifically binds to a TRAM polypeptide that includes at least one of cysteine 117 or proline 116 of SEQ ID NO: 3, or the myristoylation site of TRAM.

34. The antibody of claim 35, wherein the antibody specifically binds to a myristoylated form of TRAM, and does not substantially bind to a non-myristoylated form.
